(19) United States
(12) Patent Application Publication
(10) Pub. No.: US 2011/0245293 A1
(43) Pub. Date: Oct. 6, 2011

(54) PHARMACEUTICAL PRODUCT COMPRISING A MUSCARINIC RECEPTOR ANTAGONIST AND A BETA-2-ADRENOCEPTOR AGONIST

(75) Inventors: Barbara G. Avitabile, Essex (GB); Nicholas Charles Ray, Essex (GB); Alan John Nadin, Essex (GB)

(73) Assignee: AstraZeneca AB, Soedertaelje (SE)

(21) Appl. No.: 13/058,781

(22) PCT Filed: Aug. 11, 2009
(86) PCT No.: PCT/SE09/50925

§ 371 Date: May 10, 2011

(30) Foreign Application Priority Data
Aug. 12, 2008 (GB) 0814728.2

(51) Int. Cl. A61K 31/439; A61P 11/00

(52) U.S. Cl. 514/305

(57) ABSTRACT

The invention provides a pharmaceutical product, kit or composition comprising a first active ingredient which is a selected muscarinic receptor antagonist selected, and a second active ingredient which is a β₂-adrenoceptor agonist, of use in the treatment of respiratory diseases such as chronic obstructive pulmonary disease and asthma.

The present invention relates to combinations of pharmaceutically active substances for use in the treatment of respiratory diseases, especially chronic obstructive pulmonary disease (COPD) and asthma.

The essential function of the lungs requires a fragile structure with enormous exposure to the environment, including pollutants, microbes, allergens, and carcinogens. Host factors, resulting from interactions of lifestyle choices and genetic composition, influence the response to this exposure. Damage or infection to the lungs can give rise to a wide range of diseases of the respiratory system (or respiratory diseases). A number of these diseases are of great public health importance. Respiratory diseases include Acute Lung Injury, Acute Respiratory Distress Syndrome (ARDS), occupational lung disease, lung cancer, tuberculosis, fibrosis, pneumoconiosis, pneumonia, emphysema, Chronic Obstructive Pulmonary Disease (COPD) and asthma.

Among the most common of the respiratory diseases is asthma. Asthma is generally defined as an inflammatory disorder of the airways with clinical symptoms arising from intermittent airflow obstruction. It is characterised clinically by paroxysms of wheezing, dyspnea and cough. It is a chronic disabling disorder that appears to be increasing in prevalence and severity. It is estimated that 15% of children and 5% of adults in the population of developed countries suffer from asthma. Therapy should therefore be aimed at controlling symptoms so that normal life is possible and at the same time provide basis for treating the underlying inflammation.

COPD is a term which refers to a large group of lung diseases which can interfere with normal breathing. Current clinical guidelines define COPD as a disease state characterized by airflow limitation that is not fully reversible. The airflow limitation is usually both progressive and associated with an abnormal inflammatory response of the lungs to noxious particles and gases. The most important contributory source of such particles and gases, at least in the western world, is tobacco smoke. COPD patients have a variety of symptoms, including cough, shortness of breath, and excessive production of sputum; such symptoms arise from dysfunction of a number of cellular compartments, including neutrophils, macrophages, and epithelial cells. The two most important conditions covered by COPD are chronic bronchitis and emphysema.

Chronic bronchitis is a long-standing inflammation of the bronchi which causes increased production of mucous and other changes. The patients' symptoms are cough and expectoration of sputum. Chronic bronchitis can lead to more frequent and severe respiratory infections, narrowing and plugging of the bronchi, difficult breathing and disability.

Emphysema is a chronic lung disease which affects the alveoli and/or the ends of the smallest bronchi. The lung loses its elasticity and therefore these areas of the lungs become enlarged. These enlarged areas trap stale air and do not effectively exchange it with fresh air. This results in difficult breathing and may result in insufficient oxygen being delivered to the blood. The predominant symptom in patients with emphysema is shortness of breath.

Therapeutic agents used in the treatment of respiratory diseases include β₂-adrenoceptor agonists. These agents (also known as beta2 (β₂)-agonists) may be used to alleviate symptoms of respiratory diseases by relaxing the bronchial smooth muscles, reducing airway obstruction, reducing lung hyperinflation and decreasing shortness of breath. Compounds currently under evaluation as once-daily β2 agonists are described in Expert Opin. Investig. Drugs 14 (7), 775-783 (2005).

A further class of therapeutic agent used in the treatment of respiratory diseases are muscarinic antagonists. Muscarinic receptors are a G-protein coupled receptor (GPCR) family having five family members M₁, M₂, M₃, M₄ and M₅. Of the five muscarinic subtypes, three (M₁, M₂ and M₃) are known to exert physiological effects on human lung tissue. Parasympathetic nerves are the main pathway for reflex bronchoconstriction in human airways and mediate airway tone by releasing acetylcholine onto muscarinic receptors. Airway tone is increased in patients with respiratory disorders such as asthma and chronic obstructive pulmonary disease (COPD), and for this reason muscarinic receptor antagonists have been developed for use in treating airway diseases. Muscarinic receptor antagonists, often called anticholinergics in clinical practice, have gained widespread acceptance as a first-line therapy for individuals with COPD, and their use has been extensively reviewed in the literature (e.g. Lee et al, Current Opinion in Pharmacology 2001, 1, 223-229).

Whilst treatment with a β₂-adrenoceptor agonist or a muscarinic antagonist can yield important benefits, the efficacy of these agents is often far from satisfactory. Moreover, in view of the complexity of respiratory diseases such as asthma and COPD, it is unlikely that any one mediator can satisfactorily treat the disease alone. Hence there is a pressing medical need for new therapies against respiratory diseases such as COPD and asthma, in particular for therapies with disease modifying potential.

The present invention provides a pharmaceutical product comprising, in combination, a first active ingredient which is a muscarinic antagonist selected from:
(R)-1-[5-((R)-Cyclohexyl-hydroxy-phenyl-methyl)-[1,3,4]oxadiazol-2-ylmethyl]-3-(4-fluoro-phenoxy)-1-azonia-bicyclo[2.2.2]octane X;
(R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane X;
(R)-3-(3-Fluoro-4-methyl-phenoxy)-1-[3-(hydroxy-diphenyl-methyl)-isoxazol-5-ylmethyl]-1-azonia-bicyclo[2.2.2]octane X;
(R)-3-(3-Fluoro-phenylsulfanyl)-1-[3-(hydroxy-diphenyl-methyl)-isoxazol-5-ylmethyl]-1-azonia-bicyclo[2.2.2]octane X;
(R)-1-[3-((R)-Cyclohexyl-hydroxy-phenyl-methyl)-isoxazol-5-ylmethyl]-3-(4-fluoro-phenoxy)-1-azonia-bicyclo[2.2.2]octane X;
   wherein X represents a pharmaceutically acceptable anion of a mono or polyvalent acid, and a second active ingredient which is a β₂-adrenoceptor agonist.

A beneficial therapeutic effect may be observed in the treatment of respiratory diseases if a muscarinic antagonist according to the present invention is used in combination with a β₂-adrenoceptor agonist. The beneficial effect may be observed when the two active substances are administered simultaneously (either in a single pharmaceutical preparation or via separate preparations), or sequentially or separately via separate pharmaceutical preparations.

The pharmaceutical product of the present invention may, for example, be a pharmaceutical composition comprising the first and second active ingredients in admixture. Alternatively, the pharmaceutical product may, for example, be a kit comprising a preparation of the first active ingredient and a preparation of the second active ingredient and, optionally, instructions for the simultaneous, sequential or separate administration of the preparations to a patient in need thereof.

The first active ingredient in the combination of the present invention is a muscarinic antagonist selected from:
(R)-1-[5-((R)-Cyclohexyl-hydroxy-phenyl-methyl)-[1,3,4]oxadiazol-2-ylmethyl]-3-(4-fluoro-phenoxy)-1-azonia-bicyclo[2.2.2]octane X;
(R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane X;
(R)-3-(3-Fluoro-4-methyl-phenoxy)-1-[3-(hydroxy-diphenyl-methyl)-isoxazol-5-ylmethyl]-1-azonia-bicyclo[2.2.2]octane X;
(R)-3-(3-Fluoro-phenylsulfanyl)-1-[3-(hydroxy-diphenyl-methyl)-isoxazol-5-ylmethyl]-1-azonia-bicyclo[2.2.2]octane X;
(R)-1-[3-((R)-Cyclohexyl-hydroxy-phenyl-methyl)-isoxazol-5-ylmethyl]-3-(4-fluoro-phenoxy)-1-azonia-bicyclo[2.2.2]octane X;
   wherein X represents a pharmaceutically acceptable anion of a mono or polyvalent acid.

The muscarinic antagonists of the invention are selected members of a novel class of compound described in co-pending application PCT/GB2008/000519 (WO 2008/099186) which display high potency to the M3 receptor. The names of the muscarinic antagonists are IUPAC names generated by the Beilstein Autonom 2000 naming package, as supplied by MDL Information Systems Inc., based on the structures depicted in the examples, and stereochemistry assigned according to the Cahn-Ingold-Prelog system. For example, the name (R)-1-[3-((R)-Cyclohexyl-hydroxy-phenyl-methyl)-isoxazol-5-ylmethyl]-3-(4-fluoro-phenoxy)-1-azonia-bicyclo[2.2.2]octane, was generated from the structure:

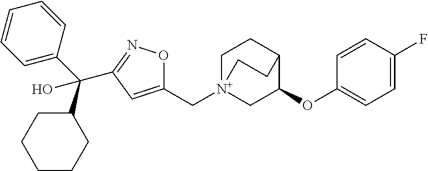

The muscarinic antagonists of the present invention comprise an anion X associated with the positive charge on the quaternary nitrogen atom. The anion X may be any pharmaceutically acceptable anion of a mono or polyvalent (e.g. bivalent) acid. In an embodiment of the invention X may be an anion of a mineral acid, for example chloride, bromide, iodide, sulfate, nitrate or phosphate; or an anion of a suitable organic acid, for example toluenesulfonate (tosylate), edisylate (ethane-1,2-disulfonate), isethionate (2-hydroxyethylsulfonate), lactate, oleic, maleate ((Z)-3-carboxy-acrylate), succinate (3-carboxy-propionate), malate ((S)-3-carboxy-2-hydroxy-propionate), p-acetamidobenzoateacetate, maleate, fumarate, citrate, oxalate, succinate, tartrate, methanesulphonate, p-toluenesulphonate, benzenesulphonate, napadisylate (naphthalene-1,5-disulphonate) (e.g. a heminapadisylate), 2,5-dichlorobenzenesulphonate, (xinafoate) 1-hydroxy-2-naphthoate or 1-hydroxynaphthalene-2-sulphonate.

In an embodiment of the invention, the muscarinic receptor antagonist is in the form of a bromide salt.

In an embodiment of the invention, the muscarinic receptor antagonist is selected from
(R)-1-[5-((R)-Cyclohexyl-hydroxy-phenyl-methyl)-[1,3,4]oxadiazol-2-ylmethyl]-3-(4-fluoro-phenoxy)-1-azonia-bicyclo[2.2.2]octane bromide;
(R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane chloride;
(R)-3-(3-Fluoro-4-methyl-phenoxy)-1-[3-(hydroxy-diphenyl-methyl)-isoxazol-5-ylmethyl]-1-azonia-bicyclo[2.2.2]octane bromide;
(R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate;
(R)-1-[3-((R)-Cyclohexyl-hydroxy-phenyl-methyl)-isoxazol-5-ylmethyl]-3-(4-fluoro-phenoxy)-1-azonia-bicyclo[2.2.2]octane benzenesulfonate;
(R)-1-[3-((R)-Cyclohexyl-hydroxy-phenyl-methyl)-isoxazol-5-ylmethyl]-3-(4-fluoro-phenoxy)-1-azonia-bicyclo[2.2.2]octane chloride; and
(R)-3-(3-Fluoro-phenylsulfanyl)-1-[3-(hydroxy-diphenyl-methyl)-isoxazol-5-ylmethyl]-1-azonia-bicyclo[2.2.2]octane bromide.

The second active ingredient in the combination of the present invention is a β₂-adrenoceptor agonist. The β₂-adrenoceptor agonist of the present invention may be any compound or substance capable of stimulating the β₂-receptors and acting as a bronchodilator. In the context of the present specification, unless otherwise stated, any reference to a β₂-adrenoceptor agonist includes active salts, solvates or derivatives that may be formed from said β₂-adrenoceptor agonist and any enantiomers and mixtures thereof. Examples of possible salts or derivatives of β₂-adrenoceptor agonist are acid addition salts such as the salts of hydrochloric acid, hydrobromic acid, sulphuric acid, phosphoric acid, methanesulphonic acid, acetic acid, fumaric acid, succinic acid, lactic acid, citric acid, tartaric acid, 1-hydroxy-2-naphthalenecarboxylic acid, maleic acid, and pharmaceutically acceptable esters (e.g. C₁-C₆ alkyl esters). The β₂-agonists may also be in the form of solvates, e.g. hydrates.

Examples of a β₂-adrenoceptor agonist that may be used in the pharmaceutical product according to this embodiment include metaproterenol, isoproterenol, isoprenaline, albuterol, salbutamol (e.g. as sulphate), formoterol (e.g. as fumarate), salmeterol (e.g. as xinafoate), terbutaline, orciprenaline, bitolterol (e.g. as mesylate), pirbuterol or indacaterol. The β₂-adrenoceptor agonist of this embodiment may be a long-acting β₂-agonist (i.e. a β₂-agonist with activity that persists for more than 24 hours), for example salmeterol (e.g. as xinafoate), formoterol (e.g. as fumarate), bambuterol (e.g. as hydrochloride), carmoterol (TA 2005, chemically identified as 2(1H)-Quinolone, 8-hydroxy-5-[1-hydroxy-2-[[2-(4-methoxy-phenyl)-1-methylethyl]-amino]ethyl]-monohydrochloride, [R-(R*,R*)] also identified by Chemical Abstract Service Registry Number 137888-11-0 and disclosed in U.S. Pat. No. 4,579,854), indacaterol (CAS no 312753-06-3; QAB-149), formanilide derivatives e.g. 3-(4-{[6-({(2R)-2-[3-(formylamino)-4-hydroxyphenyl]-2-hydroxyethyl}amino)hexyl]oxy}-butyl)-benzenesulfonamide as disclosed in WO 2002/76933, benzenesulfonamide derivatives e.g. 3-(4-{[6-({(2R)-2-hydroxy-2-[4-hydroxy-3-(hydroxy-methyl)phenyl]ethyl}amino)-hexyl]oxy}butyl)benzenesulfonamide as disclosed in WO 2002/88167, aryl aniline receptor agonists as disclosed in WO 2003/042164 and WO 2005/025555, indole derivatives as disclosed in WO 2004/032921, in US 2005/222144, compounds GSK 159797, GSK 159802, GSK 597901, GSK 642444 and GSK 678007.

In an embodiment of the present invention, the β₂-adrenoceptor agonist is formoterol. The chemical name for formoterol is N-[2-hydroxy-5-[(1)-1-hydroxy-2-[[(1)-2-(4-methoxyphenyl)-1-methylethyl]amino]ethyl]phenyl]-formamide. The preparation of formoterol is described, for example, in WO 92/05147. In one aspect of this embodiment, the β₂-adrenoceptor agonist is formoterol fumarate. It will be understood that the invention encompasses the use of all optical isomers of formoterol and mixtures thereof including racemates. Thus for example, the term formoterol encompasses N-[2-hydroxy-5-[(1R)-1-hydroxy-2-[[(1R)-2-(4-methoxyphenyl)-1-methylethyl]amino]ethyl]phenyl]-formamide, N-[2-hydroxy-5-[(1S)-1-hydroxy-2-[[(1S)-2-(4-methoxyphenyl)-1-methylethyl]amino]ethyl]phenyl]-formamide and a mixture of such enantiomers, including a racemate.

In an embodiment of the invention, the β₂-adrenoceptor agonist is selected from:
N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide,
N-[2-(Diethylamino)ethyl]-N-(2-{[2-(4-hydroxy-2-oxo-2,3-dihydro-1,3-benzothiazol-7-yl)ethyl]amino}ethyl)-3-[2-(3-chlorophenyl)ethoxy]propanamide, and
7-[(1R)-2-({2-[(3-{[2-(2-Chlorophenyl)ethyl]amino}propyl)thio]ethyl}amino)-1-hydroxyethyl]-4-hydroxy-1,3-benzothiazol-2(3H)-one,
   or a pharmaceutically acceptable salt thereof. The β₂-adrenoceptor agonists according to this embodiment may be prepared as described in the experimental preparation section of the present application. The names of the β₂-adrenoceptor agonists of this embodiment are IUPAC names generated by the IUPAC NAME, ACD Labs Version 8 naming package.

In a further embodiment of the invention, the β₂-adrenoceptor agonist is selected from:
N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide dihydrobromide,
N-[2-(Diethylamino)ethyl]-N-(2-{[2-(4-hydroxy-2-oxo-2,3-dihydro-1,3-benzothiazol-7-yl)ethyl]amino}ethyl)-3-[2-(3-chlorophenyl)ethoxy]propanamide dihydrobromide, and
7-[(1R)-2-({2-[(3-{[2-(2-Chlorophenyl)ethyl]amino}propyl)thio]ethyl}amino)-1-hydroxyethyl]-4-hydroxy-1,3-benzothiazol-2(3H)-one dihydrobromide.

In an embodiment of the invention, the muscarinic receptor antagonist is (R)-1-[3-((R)-Cyclohexyl-hydroxy-phenyl-methyl)-isoxazol-5-ylmethyl]-3-(4-fluoro-phenoxy)-1-azonia-bicyclo[2.2.2]octane X, wherein X represents a pharmaceutically acceptable anion of a mono or polyvalent acid, and the β₂-adrenoceptor agonist is formoterol (e.g. as fumarate). In one aspect of this embodiment, the muscarinic receptor antagonist is (R)-1-[3-((R)-Cyclohexyl-hydroxy-phenyl-methyl)-isoxazol-5-ylmethyl]-3-(4-fluoro-phenoxy)-1-azonia-bicyclo[2.2.2]octane chloride. In another aspect of this embodiment, the muscarinic receptor antagonist is (R)-1-[3-((R)-Cyclohexyl-hydroxy-phenyl-methyl)-isoxazol-5-ylmethyl]-3-(4-fluoro-phenoxy)-1-azonia-bicyclo[2.2.2]octane benzenesulfonate.

In an embodiment of the invention, the muscarinic receptor antagonist is (R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane X, wherein X represents a pharmaceutically acceptable anion of a mono or polyvalent acid, and the β₂-adrenoceptor agonist is formoterol (e.g. as fumarate). In one aspect of this embodiment, the muscarinic receptor antagonist is (R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane chloride. In another aspect of this embodiment, the muscarinic receptor antagonist is (R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate.

In an embodiment of the invention, the muscarinic receptor antagonist is (R)-1-[5-((R)-Cyclohexyl-hydroxy-phenyl-methyl)-[1,3,4]oxadiazol-2-ylmethyl]-3-(4-fluoro-phenoxy)-1-azonia-bicyclo[2.2.2]octane X, wherein X represents a pharmaceutically acceptable anion of a mono or polyvalent acid, and the β₂-adrenoceptor agonist is formoterol (e.g. as fumarate). In one aspect of this embodiment, the muscarinic receptor antagonist is (R)-1-[5-((R)-Cyclohexyl-hydroxy-phenyl-methyl)-[1,3,4]oxadiazol-2-ylmethyl]-3-(4-fluoro-phenoxy)-1-azonia-bicyclo[2.2.2]octane bromide.

In an embodiment of the invention, the muscarinic receptor antagonist is (R)-3-(3-Fluoro-phenylsulfanyl)-1-[3-(hydroxy-diphenyl-methyl)-isoxazol-5-ylmethyl]-1-azonia-bicyclo[2.2.2]octane X, wherein X represents a pharmaceutically acceptable anion of a mono or polyvalent acid, and the β₂-adrenoceptor agonist is formoterol (e.g. as fumarate). In one aspect of this embodiment, the muscarinic receptor antagonist is (R)-3-(3-Fluoro-phenylsulfanyl)-1-[3-(hydroxy-diphenyl-methyl)-isoxazol-5-ylmethyl]-1-azonia-bicyclo[2.2.2]octane bromide.

In an embodiment of the invention, the muscarinic receptor antagonist is (R)-3-(3-Fluoro-4-methyl-phenoxy)-1-[3-(hydroxy-diphenyl-methyl)-isoxazol-5-ylmethyl]-1-azonia-bicyclo[2.2.2]octane X, wherein X represents a pharmaceutically acceptable anion of a mono or polyvalent acid, and the β₂-adrenoceptor agonist is formoterol (e.g. as fumarate). In one aspect of this embodiment, the muscarinic receptor antagonist is (R)-3-(3-Fluoro-4-methyl-phenoxy)-1-[3-(hydroxy-diphenyl-methyl)-isoxazol-5-ylmethyl]-1-azonia-bicyclo[2.2.2]octane bromide.

In an embodiment of the invention, the muscarinic receptor antagonist is (R)-1-[3-((R)-Cyclohexyl-hydroxy-phenyl-methyl)-isoxazol-5-ylmethyl]-3-(4-fluoro-phenoxy)-1-azonia-bicyclo[2.2.2]octane X, wherein X represents a pharmaceutically acceptable anion of a mono or polyvalent acid, and the β₂-adrenoceptor agonist is N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide or a pharmaceutically acceptable salt thereof (e.g. dihydrobromide). In one aspect of this embodiment, the muscarinic receptor antagonist is (R)-1-[3-((R)-Cyclohexyl-hydroxy-phenyl-methyl)-isoxazol-5-ylmethyl]-3-(4-fluoro-phenoxy)-1-azonia-bicyclo[2.2.2]octane chloride. In another aspect of this embodiment, the muscarinic receptor antagonist is (R)-1-[3-((R)-Cyclohexyl-hydroxy-phenyl-methyl)-isoxazol-5-ylmethyl]-3-(4-fluoro-phenoxy)-1-azonia-bicyclo[2.2.2]octane benzenesulfonate.

In an embodiment of the invention, the muscarinic receptor antagonist is (R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane X, wherein X represents a pharmaceutically acceptable anion of a mono or polyvalent acid, and the β₂-adrenoceptor agonist is N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide or a pharmaceutically acceptable salt thereof (e.g. dihydrobromide). In one aspect of this embodiment, the muscarinic receptor antagonist is (R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane chloride. In another aspect of this embodiment, the muscarinic receptor antagonist is (R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate. In another aspect of this embodiment, the muscarinic receptor antagonist is (R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane 2-hydroxy-ethane sulfonate.

In an embodiment of the invention, the muscarinic receptor antagonist is (R)-1-[5-((R)-Cyclohexyl-hydroxy-phenyl-methyl)-[1,3,4]oxadiazol-2-ylmethyl]-3-(4-fluoro-phenoxy)-1-azonia-bicyclo[2.2.2]octane X, wherein X represents a pharmaceutically acceptable anion of a mono or polyvalent acid, and the β₂-adrenoceptor agonist is N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide or a pharmaceutically acceptable salt thereof (e.g. dihydrobromide). In one aspect of this embodiment, the muscarinic receptor antagonist is (R)-1-[5-((R)-Cyclohexyl-hydroxy-phenyl-methyl)-[1,3,4]oxadiazol-2-ylmethyl]-3-(4-fluoro-phenoxy)-1-azonia-bicyclo[2.2.2]octane bromide.

In an embodiment of the invention, the muscarinic receptor antagonist is (R)-3-(3-Fluoro-phenylsulfanyl)-1-[3-(hydroxy-diphenyl-methyl)-isoxazol-5-ylmethyl]-1-azonia-bicyclo[2.2.2]octane X, wherein X represents a pharmaceutically acceptable anion of a mono or polyvalent acid, and the β₂-adrenoceptor agonist is N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide or a pharmaceutically acceptable salt thereof (e.g. dihydrobromide). In one aspect of this embodiment, the muscarinic receptor antagonist is (R)-3-(3-Fluoro-phenylsulfanyl)-1-[3-(hydroxy-diphenyl-methyl)-isoxazol-5-ylmethyl]-1-azonia-bicyclo[2.2.2]octane bromide.

In an embodiment of the invention, the muscarinic receptor antagonist is (R)-3-(3-Fluoro-4-methyl-phenoxy)-1-[3-(hydroxy-diphenyl-methyl)-isoxazol-5-ylmethyl]-1-azonia-bicyclo[2.2.2]octane X, wherein X represents a pharmaceutically acceptable anion of a mono or polyvalent acid, and the β₂-adrenoceptor agonist is N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide or a pharmaceutically acceptable salt thereof (e.g. dihydrobromide). In one aspect of this embodiment, the muscarinic receptor antagonist is (R)-3-(3-Fluoro-4-methyl-phenoxy)-1-[3-(hydroxy-diphenyl-methyl)-isoxazol-5-ylmethyl]-1-azonia-bicyclo[2.2.2]octane bromide.

In an embodiment of the invention, the muscarinic receptor antagonist is (R)-1-[3-((R)-Cyclohexyl-hydroxy-phenyl-methyl)-isoxazol-5-ylmethyl]-3-(4-fluoro-phenoxy)-1-azonia-bicyclo[2.2.2]octane X, wherein X represents a pharmaceutically acceptable anion of a mono or polyvalent acid, and the β₂-adrenoceptor agonist is 7-[(1R)-2-({2-[(3-{[2-(2-Chlorophenyl)ethyl]amino}propyl)thio]ethyl}amino)-1-hydroxyethyl]-4-hydroxy-1,3-benzothiazol-2(3H)-one or a pharmaceutically acceptable salt thereof (e.g. dihydrobromide). In one aspect of this embodiment, the muscarinic receptor antagonist is (R)-1-[3-((R)-Cyclohexyl-hydroxy-phenyl-methyl)-isoxazol-5-ylmethyl]-3-(4-fluoro-phenoxy)-1-azonia-bicyclo[2.2.2]octane chloride. In another aspect of this embodiment, the muscarinic receptor antagonist is (R)-1-[3-((R)-Cyclohexyl-hydroxy-phenyl-methyl)-isoxazol-5-ylmethyl]-3-(4-fluoro-phenoxy)-1-azonia-bicyclo[2.2.2]octane benzenesulfonate.

In an embodiment of the invention, the muscarinic receptor antagonist is (R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane X, wherein X represents a pharmaceutically acceptable anion of a mono or polyvalent acid, and the β₂-adrenoceptor agonist is 7-[(1R)-2-({2-[(3-{[2-(2-Chlorophenyl)ethyl]amino}propyl)thio]ethyl}amino)-1-hydroxyethyl]-4-hydroxy-1,3-benzothiazol-2(3H)-one or a pharmaceutically acceptable salt thereof (e.g. dihydrobromide). In one aspect of this embodiment, the muscarinic receptor antagonist is (R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane chloride. In another aspect of this embodiment, the muscarinic receptor antagonist is (R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate.

In an embodiment of the invention, the muscarinic receptor antagonist is (R)-1-[5-((R)-Cyclohexyl-hydroxy-phenyl-methyl)-[1,3,4]oxadiazol-2-ylmethyl]-3-(4-fluoro-phenoxy)-1-azonia-bicyclo[2.2.2]octane X, wherein X represents a pharmaceutically acceptable anion of a mono or polyvalent acid, and the β₂-adrenoceptor agonist is 7-[(1R)-2-({2-[(3-{[2-(2-Chlorophenyl)ethyl]amino}propyl)thio]ethyl}amino)-1-hydroxyethyl]-4-hydroxy-1,3-benzothiazol-2(3H)-one or a pharmaceutically acceptable salt thereof (e.g. dihydrobromide). In one aspect of this embodiment, the muscarinic receptor antagonist is (R)-1-[5-((R)-Cyclohexyl-hydroxy-phenyl-methyl)-[1,3,4]oxadiazol-2-ylmethyl]-3-(4-fluoro-phenoxy)-1-azonia-bicyclo[2.2.2]octane bromide.

In an embodiment of the invention, the muscarinic receptor antagonist is (R)-3-(3-Fluoro-phenylsulfanyl)-1-[3-(hydroxy-diphenyl-methyl)-isoxazol-5-ylmethyl]-1-azonia-bicyclo[2.2.2]octane X, wherein X represents a pharmaceutically acceptable anion of a mono or polyvalent acid, and the β₂-adrenoceptor agonist is 7-[(1R)-2-({2-[(3-{[2-(2-Chlorophenyl)ethyl]amino}propyl)thio]ethyl}amino)-1-hydroxyethyl]-4-hydroxy-1,3-benzothiazol-2(3H)-one or a pharmaceutically acceptable salt thereof (e.g. dihydrobromide). In one aspect of this embodiment, the muscarinic receptor antagonist is (R)-3-(3-Fluoro-phenylsulfanyl)-1-[3-(hydroxy-diphenyl-methyl)-isoxazol-5-ylmethyl]-1-azonia-bicyclo[2.2.2]octane bromide.

In an embodiment of the invention, the muscarinic receptor antagonist is (R)-3-(3-Fluoro-4-methyl-phenoxy)-1-[3-(hydroxy-diphenyl-methyl)-isoxazol-5-ylmethyl]-1-azonia-bicyclo[2.2.2]octane X, wherein X represents a pharmaceutically acceptable anion of a mono or polyvalent acid, and the β₂-adrenoceptor agonist is 7-[(1R)-2-({2-[(3-{[2-(2-Chlorophenyl)ethyl]amino}propyl)thio]ethyl}amino)-1-hydroxyethyl]-4-hydroxy-1,3-benzothiazol-2(3H)-one or a pharmaceutically acceptable salt thereof (e.g. dihydrobromide). In one aspect of this embodiment, the muscarinic receptor antagonist is (R)-3-(3-Fluoro-4-methyl-phenoxy)-1-[3-(hydroxy-diphenyl-methyl)-isoxazol-5-ylmethyl]-1-azonia-bicyclo[2.2.2]octane bromide.

In an embodiment of the invention, the β₂-adrenoceptor agonist is N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide or a pharmaceutically acceptable salt thereof. The β₂-adrenoceptor agonist according to this embodiment may be prepared as described in WO2008/075026 A1. In a further aspect of this embodiment, the β₂-adrenoceptor agonist is N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide bis-trifluoroacetic acid salt. In a further aspect of this embodiment, the β₂-adrenoceptor agonist is N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide dihydrobromide salt. In a further aspect of this embodiment, the β₂-adrenoceptor agonist is N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide di-D-mandelate salt.

In an embodiment of the invention, the muscarinic receptor antagonist is (R)-1-[3-((R)-Cyclohexyl-hydroxy-phenyl-methyl)-isoxazol-5-ylmethyl]-3-(4-fluoro-phenoxy)-1-azonia-bicyclo[2.2.2]octane X, wherein X represents a pharmaceutically acceptable anion of a mono or polyvalent acid, and the β₂-adrenoceptor agonist is N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide or a pharmaceutically acceptable salt thereof (e.g. dihydrobromide or di-D-mandelate salt). In one aspect of this embodiment, the muscarinic receptor antagonist is (R)-1-[3-((R)-Cyclohexyl-hydroxy-phenyl-methyl)-isoxazol-5-ylmethyl]-3-(4-fluoro-phenoxy)-1-azonia-bicyclo[2.2.2]octane chloride. In another aspect of this embodiment, the muscarinic receptor antagonist is (R)-1-[3-((R)-Cyclohexyl-hydroxy-phenyl-methyl)-isoxazol-5-ylmethyl]-3-(4-fluoro-phenoxy)-1-azonia-bicyclo[2.2.2]octane benzenesulfonate.

In an embodiment of the invention, the muscarinic receptor antagonist is (R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane X, wherein X represents a pharmaceutically acceptable anion of a mono or polyvalent acid, and the β₂-adrenoceptor agonist is N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide or a pharmaceutically acceptable salt thereof (e.g. dihydrobromide or di-D-mandelate salt). In one aspect of this embodiment, the muscarinic receptor antagonist is (R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane chloride. In another aspect of this embodiment, the muscarinic receptor antagonist is (R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane 2-hydroxy-ethane sulfonate.

In an embodiment of the invention, the muscarinic receptor antagonist is (R)-1-[5-((R)-Cyclohexyl-hydroxy-phenyl-methyl)-[1,3,4]oxadiazol-2-ylmethyl]-3-(4-fluoro-phenoxy)-1-azonia-bicyclo[2.2.2]octane X, wherein X represents a pharmaceutically acceptable anion of a mono or polyvalent acid, and the β₂-adrenoceptor agonist is N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide or a pharmaceutically acceptable salt thereof (e.g. dihydrobromide or di-D-mandelate salt). In one aspect of this embodiment, the muscarinic receptor antagonist is (R)-1-[5-((R)-Cyclohexyl-hydroxy-phenyl-methyl)-[1,3,4]oxadiazol-2-ylmethyl]-3-(4-fluoro-phenoxy)-1-azonia-bicyclo[2.2.2]octane bromide.

In an embodiment of the invention, the muscarinic receptor antagonist is (R)-3-(3-Fluoro-phenylsulfanyl)-1-[3-(hydroxy-diphenyl-methyl)-isoxazol-5-ylmethyl]-1-azonia-bicyclo[2.2.2]octane X, wherein X represents a pharmaceutically acceptable anion of a mono or polyvalent acid, and the β₂-adrenoceptor agonist is N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide or a pharmaceutically acceptable salt thereof (e.g. dihydrobromide or di-D-mandelate salt). In one aspect of this embodiment, the muscarinic receptor antagonist is (R)-3-(3-Fluoro-phenylsulfanyl)-1-[3-(hydroxy-diphenyl-methyl)-isoxazol-5-ylmethyl]-1-azonia-bicyclo[2.2.2]octane bromide.

In an embodiment of the invention, the muscarinic receptor antagonist is (R)-3-(3-Fluoro-4-methyl-phenoxy)-1-[3-(hydroxy-diphenyl-methyl)-isoxazol-5-ylmethyl]-1-azonia-bicyclo[2.2.2]octane X, wherein X represents a pharmaceutically acceptable anion of a mono or polyvalent acid, and the β₂-adrenoceptor agonist is N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide or a pharmaceutically acceptable salt thereof (e.g. dihydrobromide or di-D-mandelate salt). In one aspect of this embodiment, the muscarinic receptor antagonist is (R)-3-(3-Fluoro-4-methyl-phenoxy)-1-[3-(hydroxy-diphenyl-methyl)-isoxazol-5-ylmethyl]-1-azonia-bicyclo[2.2.2]octane bromide.

The combination of the present invention may provide a beneficial therapeutic effect in the treatment of respiratory diseases. Examples of such possible effects include improvements in one or more of the following parameters: reducing inflammatory cell influx into the lung, mild and severe exacerbations, FEV₁ (forced expiratory volume in one second), vital capacity (VC), peak expiratory flow (PEF), symptom scores and Quality of Life.

The muscarinic antagonist (first active ingredient) and β₂-adrenoceptor agonist (second active ingredient) of the present invention may be administered simultaneously, sequentially or separately to treat respiratory diseases. By sequential it is meant that the active ingredients are administered, in any order, one immediately after the other. They may still have the desired effect if they are administered separately, but when administered in this manner they will generally be administered less than 4 hours apart, more conveniently less than two hours apart, more conveniently less than 30 minutes apart and most conveniently less than 10 minutes apart.

The active ingredients of the present invention may be administered by oral or parenteral (e.g. intravenous, subcutaneous, intramuscular or intraarticular) administration using conventional systemic dosage forms, such as tablets, capsules, pills, powders, aqueous or oily solutions or suspensions, emulsions and sterile injectable aqueous or oily solutions or suspensions. The active ingredients may also be administered topically (to the lung and/or airways) in the form of solutions, suspensions, aerosols and dry powder. These dosage forms will usually include one or more pharmaceutically acceptable ingredients which may be selected, for example, from adjuvants, carriers, binders, lubricants, diluents, stabilising agents, buffering agents, emulsifying agents, viscosity-regulating agents, surfactants, preservatives, flavourings and colorants. As will be understood by those skilled in the art, the most appropriate method of administering the active ingredients is dependent on a number of factors.

In one embodiment of the present invention the active ingredients are administered via separate pharmaceutical preparations. Therefore, in one aspect, the present invention provides a kit comprising a preparation of a first active ingredient which is a muscarinic antagonist according to the present invention, and a preparation of a second active ingredient which is a β₂-adrenoceptor agonist, and optionally instructions for the simultaneous, sequential or separate administration of the preparations to a patient in need thereof.

In another embodiment the active ingredients may be administered via a single pharmaceutical composition. Therefore, the present invention further provides a pharmaceutical composition comprising, in admixture, a first active ingredient, which is a muscarinic antagonist according to the present invention, and a second active ingredient, which is a β₂-adrenoceptor agonist.

The pharmaceutical compositions of the present invention may be prepared by mixing the muscarinic antagonist (first active ingredient) with a β₂-adrenoceptor agonist (second active ingredient) and a pharmaceutically acceptable adjuvant, diluent or carrier. Therefore, in a further aspect of the present invention there is provided a process for the preparation of a pharmaceutical composition, which comprises mixing a muscarinic antagonist according to the present invention with a β₂-adrenoceptor agonist and a pharmaceutically acceptable adjuvant, diluent or carrier.

It will be understood that the therapeutic dose of each active ingredient administered in accordance with the present invention will vary depending upon the particular active ingredient employed, the mode by which the active ingredient is to be administered, and the condition or disorder to be treated.

In one embodiment of the present invention, the muscarinic antagonist according to the present invention is administered via inhalation. When administered via inhalation the dose of the muscarinic antagonist according to the present invention will generally be in the range of from 0.1 microgram (μg) to 5000 μg, 0.1 to 1000 μg, 0.1 to 500 μg, 0.1 to 100 μg, 0.1 to 50 μg, 0.1 to 5 μg, 5 to 5000 μg, 5 to 1000 μg, 5 to 500 μg, 5 to 100 μg, 5 to 50 μg, 5 to 10 μg, 10 to 5000 μg, 10 to 1000 μg, 10 to 500 μg, 10 to 100 μg, 10 to 50 μg, 20 to 5000 μg, 20 to 1000 μg, 20 to 500 μg, 20 to 100 μg, 20 to 50 μg, 50 to 5000 μg, 50 to 1000 μg, 50 to 500 μg, 50 to 100 μg, 100 to 5000 μg, 100 to 1000 μg or 100 to 500 μg. The dose will generally be administered from 1 to 4 times a day, conveniently once or twice a day, and most conveniently once a day.

In one embodiment of the present invention the β₂-adrenoceptor agonist may conveniently be administered by inhalation. When administered via inhalation the dose of the β₂-adrenoceptor agonist will generally be in the range of from 0.1 to 50 μg, 0.1 to 40 μg, 0.1 to 30 μg, 0.1 to 20 μg, 0.1 to 10 μg, 5 to 10 μg, 5 to 50 μg, 5 to 40 μg, 5 to 30 μg, 5 to 20 μg, 5 to 10 μg, 10 to 50 μg, 10 to 40 μg 10 to 30 μg, or 10 to 20 μg. The dose will generally be administered from 1 to 4 times a day, conveniently once or twice a day, and most conveniently once a day.

In one embodiment, the present invention provides a pharmaceutical product comprising, in combination, a first active ingredient which is a muscarinic antagonist, and a second active ingredient which is a β₂-adrenoceptor agonist, wherein each active ingredient is formulated for inhaled administration.

In an embodiment the pharmaceutical preparations of active ingredients may be administered simultaneously.

In an embodiment the different pharmaceutical preparations of active ingredients may be administered sequentially.

In an embodiment the different pharmaceutical preparations of active ingredients may be administered separately.

The active ingredients of the present invention are conveniently administered via inhalation (e.g. topically to the lung and/or airways) in the form of solutions, suspensions, aerosols and dry powder formulations. For example metered dose inhaler devices may be used to administer the active ingredients, dispersed in a suitable propellant and with or without additional excipients such as ethanol, surfactants, lubricants or stabilising agents. Suitable propellants include hydrocarbon, chlorofluorocarbon and hydrofluoroalkane (e.g. heptafluoroalkane) propellants, or mixtures of any such propellants. Preferred propellants are P134a and P227, each of which may be used alone or in combination with other propellants and/or surfactant and/or other excipients. Nebulised aqueous suspensions or, preferably, solutions may also be employed, with or without a suitable pH and/or tonicity adjustment, either as a unit-dose or multi-dose.

Dry powders and pressurized HFA aerosols of the active ingredients may be administered by oral or nasal inhalation. For inhalation, the compound is desirably finely divided. The finely divided compound preferably has a mass median diameter of less than 10 μm, and may be suspended in a propellant mixture with the assistance of a dispersant, such as a C₈-C₂₀ fatty acid or salt thereof, (for example, oleic acid), a bile salt, a phospholipid, an alkyl saccharide, a perfluorinated or polyethoxylated surfactant, or other pharmaceutically acceptable dispersant.

One possibility is to mix the finely divided compound of the invention with a carrier substance, for example, a mono-, di- or polysaccharide, a sugar alcohol, or another polyol. Suitable carriers are sugars, for example, lactose, glucose, raffinose, melezitose, lactitol, maltitol, trehalose, sucrose, mannitol; and starch. Alternatively the finely divided compound may be coated by another substance. The powder mixture may also be dispensed into hard gelatine capsules, each containing the desired dose of the active compound.

Another possibility is to process the finely divided powder into spheres which break up during the inhalation procedure. This spheronized powder may be filled into the drug reservoir of a multidose inhaler, for example, that known as the Turbuhaler® in which a dosing unit meters the desired dose which is then inhaled by the patient. With this system the active ingredient, with or without a carrier substance, is delivered to the patient.

The combination of the present invention is useful in the treatment or prevention of respiratory-tract disorders such as chronic obstructive pulmonary disease (COPD), chronic bronchitis of all types (including dyspnoea associated therewith), asthma (allergic and non-allergic; ‘wheezy-infant syndrome’), adult/acute respiratory distress syndrome (ARDS), chronic respiratory obstruction, bronchial hyperactivity, pulmonary fibrosis, pulmonary emphysema, and allergic rhinitis, exacerbation of airway hyperreactivity consequent to other drug therapy, particularly other inhaled drug therapy or pneumoconiosis (for example aluminosis, anthracosis, asbestosis, chalicosis, ptilosis, siderosis, silicosis, tabacosis and byssinosis).

Dry powder inhalers may be used to administer the active ingredients, alone or in combination with a pharmaceutically acceptable carrier, in the later case either as a finely divided powder or as an ordered mixture. The dry powder inhaler may be single dose or multi-dose and may utilise a dry powder or a powder-containing capsule.

Metered dose inhaler, nebuliser and dry powder inhaler devices are well known and a variety of such devices are available.

The present invention further provides a pharmaceutical product, kit or pharmaceutical composition according to the invention for simultaneous, sequential or separate use in therapy.

The present invention further provides the use of a pharmaceutical product, kit or pharmaceutical composition according to the invention in the manufacture of a medicament for the treatment of a respiratory disease, in particular chronic obstructive pulmonary disease or asthma.

The present invention further provides a pharmaceutical product, kit or pharmaceutical composition according to the invention for use in the treatment of a respiratory disease, in particular chronic obstructive pulmonary disease or asthma.

The present invention still further provides a method of treating a respiratory disease which comprises simultaneously, sequentially or separately administering:

(a) a (therapeutically effective) dose of a first active ingredient which is a muscarinic antagonist according to the present invention; and
(b) a (therapeutically effective) dose of a second active which is a β₂-adrenoceptor agonist;
to a patient in need thereof.

In the context of the present specification, the term “therapy” also includes “prophylaxis” unless there are specific indications to the contrary. The terms “therapeutic” and “therapeutically” should be construed accordingly. Prophylaxis is expected to be particularly relevant to the treatment of persons who have suffered a previous episode of, or are otherwise considered to be at increased risk of, the condition or disorder in question. Persons at risk of developing a particular condition or disorder generally include those having a family history of the condition or disorder, or those who have been identified by genetic testing or screening to be particularly susceptible to developing the condition or disorder.

The term “disease, unless stated otherwise, has the same meaning as the terms “condition” and “disorder” and are used interchangeably throughout the description and claims. The term “agent” and “active ingredient” means the compounds comprised in the combination of the present invention, e.g. a muscarinc antagonist or a β₂-adrenoceptor agonist.

The pharmaceutical product, kit or composition of the present invention may optionally comprise a third active ingredient which third active ingredient is a substance suitable for use in the treatment of respiratory diseases. Examples of third active ingredients that may be incorporated into the present invention include
a phosphodiesterase inhibitor,
a modulator of chemokine receptor function,
an inhibitor of kinase function,
a protease inhibitor,
a steroidal glucocorticoid receptor agonist, and
a non-steroidal glucocorticoid receptor agonist.

Examples of a phosphodiesterase inhibitor that may be used as a third active ingredient according to this embodiment include a PDE4 inhibitor such as an inhibitor of the isoform PDE4D, a PDE3 inhibitor and a PDE5 inhibitor. Examples include the compounds
(Z)-3-(3,5-dichloro-4-pyridyl)-2-[4-(2-indanyloxy-5-methoxy-2-pyridyl]propenenitrile, N-[9-amino-4-oxo-1-phenyl-3,4,6,7-tetrahydropyrrolo[3,2,1-jk][1,4]benzodiazepin-3(R)-yl]pyridine-3-carboxamide (CI-1044),
3-(benzyloxy)-1-(4-fluorobenzyl)-N-[3-(methylsulphonyl)phenyl]-1H-indole-2-carboxamide,
(1S-exo)-5-[3-(bicyclo[2.2.1]hept-2-yloxy)-4-methoxyphenyl]tetrahydro-2(1H)-pyrimidinone (Atizoram),
N-(3,5,dichloro-4-pyridinyl)-2-[1-(4-fluorobenzyl)-5-hydroxy-1H-indol-3-yl]-2-oxoacetamide (AWD-12-281),
β-[3-(cyclopentyloxy)-4-methoxyphenyl]-1,3-dihydro-1,3-dioxo-2H-isoindole-2-propanamide (CDC-801),
N-[9-methyl-4-oxo-1-phenyl-3,4,6,7-tetrahydropyrrolo[3,2,1-jk][1,4]benzodiazepin-3(R)-yl]pyridine-4-carboxamide (CI-1018),
cis-[4-cyano-4-(3-cyclopentyloxy-4-methoxyphenyl)cyclohexane-1-carboxylic acid (Cilomilast),
8-amino-1,3-bis(cyclopropylmethyl)xanthine (Cipamfylline),
N-(2,5-dichloro-3-pyridinyl)-8-methoxy-5-quinolinecarboxamide (D-4418),
5-(3,5-di-tert-butyl-4-hydroxybenzylidene)-2-iminothiazolidin-4-one (Darbufelone),
2-methyl-1-[2-(1-methylethyl)pyrazolo[1,5-a]pyridin-3-yl]-1-propanone (Ibudilast),
2-(2,4-dichlorophenylcarbonyl)-3-ureidobenzofuran-6-ylmethanesulphonate (Lirimilast),
(−)-(R)-5-(4-methoxy-3-propoxyphenyl)-5-methyloxazolidin-2-one (Mesopram),
(−)-cis-9-ethoxy-8-methoxy-2-methyl-1,2,3,4,4a,10b-hexahydro-6-(4-diisopropylaminocarbonylphenyl)-benzo[c][1,6]naphthyridine (Pumafentrine),
3-(cyclopropylmethoxy)-N-(3,5-dichloro-4-pyridyl)-4-(difluoromethoxy)benzamide (Roflumilast),
the N-oxide of Roflumilast,
5,6-diethoxybenzo[b]thiophene-2-carboxylic acid (Tibenelast),
2,3,6,7-tetrahydro-2-(mesitylimino)-9,10-dimethoxy-3-methyl-4H-pyrimido[6,1-a]isoquinolin-4-one (trequinsin), and
3-[[3-(cyclopentyloxy)-4-methoxyphenyl]-methyl]-N-ethyl-8-(1-methylethyl)-3H-purine-6-amine (V-11294A).

Examples of a modulator of chemokine receptor function that may be used as a third active ingredient according to this embodiment include a CCR3 receptor antagonist, a CCR4 receptor antagonist, a CCR5 receptor antagonist and a CCR8 receptor antagonist.

Examples of an inhibitor of kinase function that may be used as a third active ingredient according to this embodiment include a p38 kinase inhibitor and an IKK inhibitor.

Examples of a protease inhibitor that may be used as a third active ingredient according to this embodiment include an inhibitor of neutrophil elastase or an inhibitor of MMP12.

Examples of a steroidal glucocorticoid receptor agonist that may be used as a third active ingredient according to this embodiment include budesonide, fluticasone (e.g. as propionate ester), mometasone (e.g. as furoate ester), beclomethasone (e.g. as 17-propionate or 17,21-dipropionate esters), ciclesonide, loteprednol (as e.g. etabonate), etiprednol (as e.g. dicloacetate), triamcinolone (e.g. as acetonide), flunisolide, zoticasone, flumoxonide, rofleponide, butixocort (e.g. as propionate ester), prednisolone, prednisone, tipredane, steroid esters e.g. 6α,9α-difluoro-17α-[(2-furanylcarbonyl)oxy]-11β-hydroxy-16α-methyl-3-oxo-androsta-1,4-diene-17β-carbothioic acid S-fluoromethyl ester, 6α,9α-difluoro-11β-hydroxy-16α-methyl-3-oxo-17α-propionyloxy-androsta-1,4-diene-17β-carbothioic acid S-(2-oxo-tetrahydro-furan-3S-yl)ester and 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester, steroid esters according to DE 4129535, steroids according to WO 2002/00679, WO 2005/041980, or steroids GSK 870086, GSK 685698 and GSK 799943.

Examples of a modulator of a non-steroidal glucocorticoid receptor agonist that may be used as a third active ingredient according to this embodiment include those described in WO2006/046916.

The invention is illustrated by the following non-limiting Examples. In the Examples the following Figures are presented:

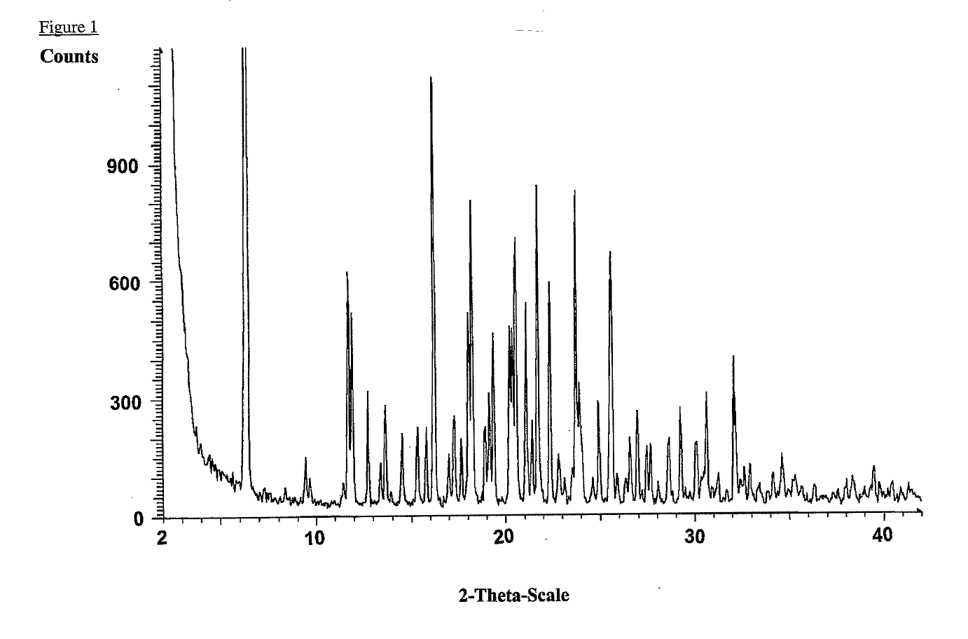

FIG. 1: X-ray powder diffraction pattern of muscarinic antagonist (R)-1-[3-((R)-Cyclohexyl-hydroxy-phenyl-methyl)-isoxazol-5-ylmethyl]-3-(4-fluoro-phenoxy)-1-azonia-bicyclo[2.2.2]octane; benzenesulfonate (Example 2).

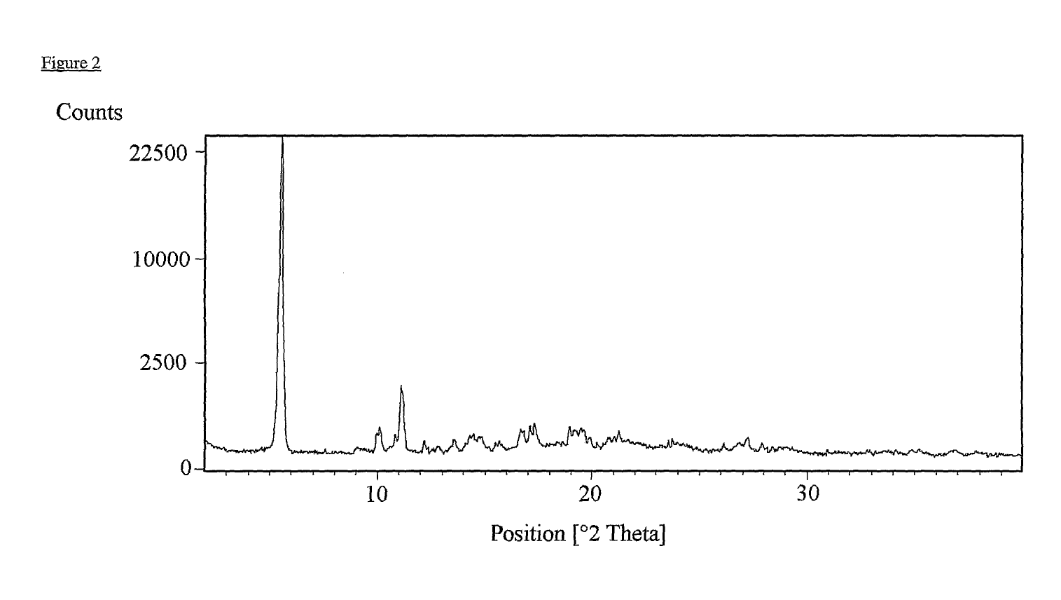

FIG. 2: X-ray powder diffraction pattern of muscarinic antagonist (R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane chloride (Example 3).

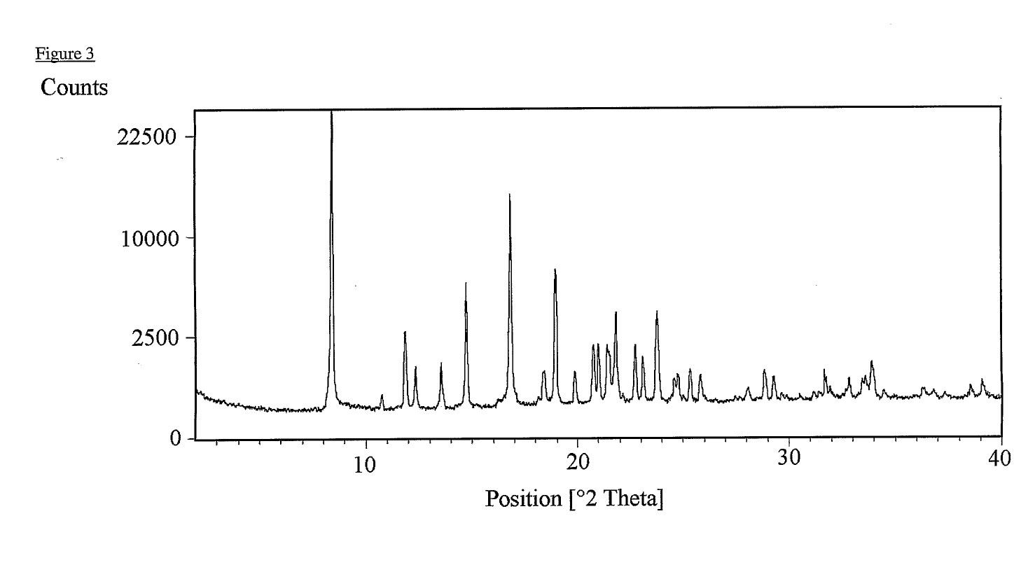

FIG. 3: X-ray powder diffraction pattern of muscarinic antagonist (R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate (Example 4).

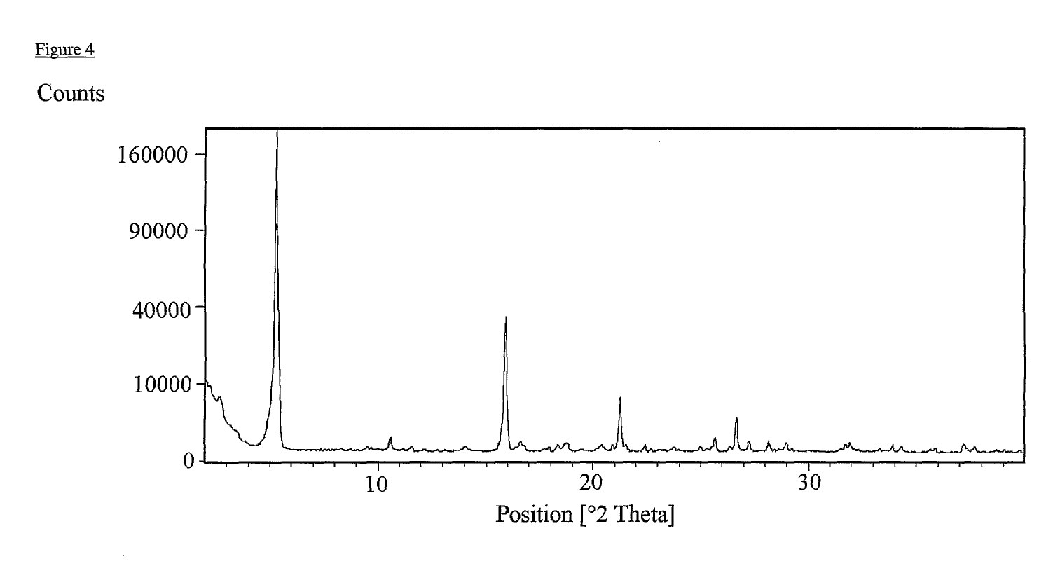

FIG. 4: X-ray powder diffraction pattern of muscarinic antagonist (R)-1-[5-((R)-Cyclohexyl-hydroxy-phenyl-methyl)-[1,3,4]oxadiazol-2-ylmethyl]-3-(4-fluoro-phenoxy)-1-azonia-bicyclo[2.2.2]octane bromide (Example 5).

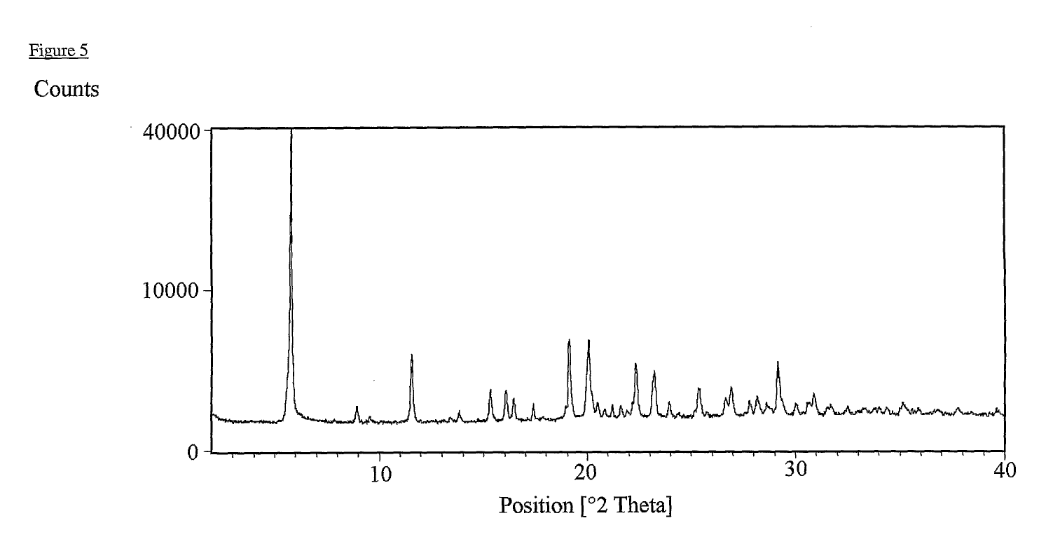

FIG. 5: X-ray powder diffraction pattern of muscarinic antagonist (R)-3-(3-Fluoro-4-methyl-phenoxy)-1-[3-(hydroxy-diphenyl-methyl)-isoxazol-5-ylmethyl]-1-azonia-bicyclo[2.2.2]octane bromide (Example 7).

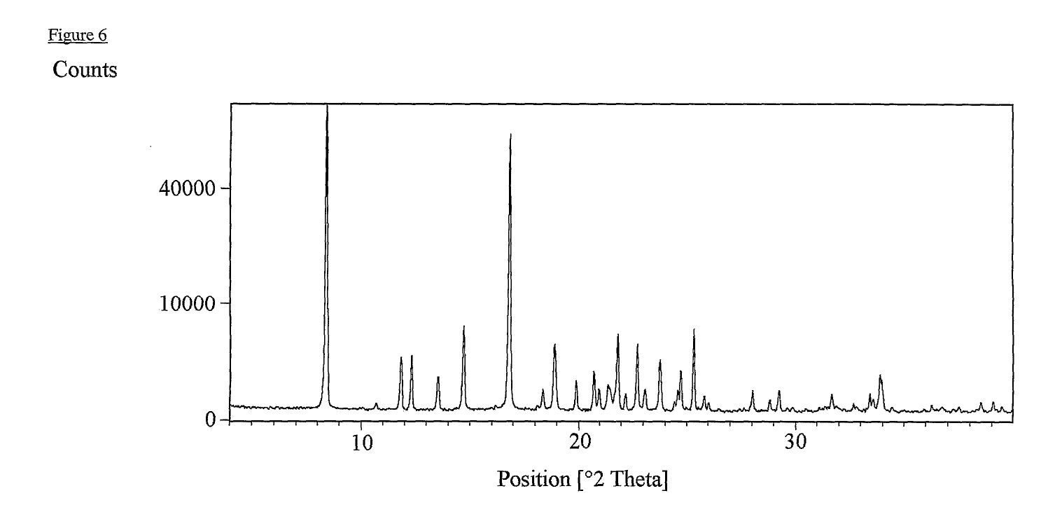

FIG. 6: X-ray powder diffraction pattern of muscarinic antagonist (R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate (Example 8).

### PREPARATION OF MUSCARINIC ANTAGONISTS

Muscarinic antagonists according to the present invention may be prepared as follows. Alternative salts to those described herein may be prepared by conventional chemistry using methods analogous to those described.

### General Experimental Details for Preparation of Muscarinic Antagonists

Unless otherwise stated the following general conditions were used in the preparation of the Muscarinic Antagonists

All reactions were carried out under an atmosphere of nitrogen unless specified otherwise. NMR spectra were obtained on a Varian Unity Inova 400 spectrometer with a 5 mm inverse detection triple resonance probe operating at 400 MHz or on a Bruker Avance DRX 400 spectrometer with a 5 mm inverse detection triple resonance TXI probe operating at 400 MHz or on a Bruker Avance DPX 300 spectrometer with a standard 5 mm dual frequency probe operating at 300 MHz. Shifts are given in ppm relative to tetramethylsilane. Where products were purified by column chromatography, ‘flash silica’ refers to silica gel for chromatography, 0.035 to 0.070 mm (220 to 440 mesh) (e.g. Fluka silica gel 60), and an applied pressure of nitrogen up to 10 p.s.i accelerated column elution or use of the semi-automated CombiFlash® Companion purification system or by manual elution of Biotage® Isolute Flash Si II cartridges under reduced pressure or by use of the Biotage® SP1 semi-automated system. All solvents and commercial reagents were used as received. SCX chromatography was performed on Biotage® Isolute SCX or SCX-2 pre-packed cartridges.

The Liquid Chromatography Mass Spectroscopy (LCMS) methods referred to are described below:

### Method 1

Waters Micromass ZQ2000 with a C18-reverse-phase column (100×3.0 mm Higgins Clipeus with 5 μm particle size), elution with A: water+0.1% formic acid; B: acetonitrile+0.1% formic acid. Gradient:

| Gradient - Time | flow mL/min | % A | % B |
|---|---|---|---|
| 0.00 | 1.0 | 95 | 5 |
| 1.00 | 1.0 | 95 | 5 |
| 15.00 | 1.0 | 5 | 95 |
| 20.00 | 1.0 | 5 | 95 |
| 22.00 | 1.0 | 95 | 5 |
| 25.00 | 1.0 | 95 | 5 |

Detection—MS, ELS, UV (100 μl split to MS with in-line UV detector) MS ionisation method—Electrospray (positive ion)

### Method 2

Waters Platform LC Quadrupole mass spectrometer with a C18-reverse-phase column (30×4.6 mm Phenomenex Luna 3 μm particle size), elution with A: water+0.1% formic acid; B: acetonitrile+0.1% formic acid. Gradient:

| Gradient - Time | flow mL/min | % A | % B |
|---|---|---|---|
| 0.00 | 2.0 | 95 | 5 |
| 0.50 | 2.0 | 95 | 5 |
| 4.50 | 2.0 | 5 | 95 |
| 5.50 | 2.0 | 5 | 95 |
| 6.00 | 2.0 | 95 | 5 |

Detection—MS, ELS, UV (200 μl split to MS with in-line UV detector) MS ionisation method—Electrospray (positive and negative ion).

Abbreviations used in the experimental section: AlBN=2,2′-azobis(2-methylpropionitrile); DCM=dichloromethane; DMF=dimethylformamide; DMSO=dimethyl sulfoxide; IMS=industrial methylated spirit; LCMS=Liquid Chromatography-Mass Spectrometry; NBS=N-bromosuccinimide; RT=room temperature; Rt=retention time; TFA=trifluoroacetic acid; THF=tetrahydrofuran; SCX=strong cation exchange chromatography.

### For the Analysis of the Crystalline Form of Example 2:

Differential Scanning calorimetry (DSC) measurements were performed on a Mettler Toledo DSC823e equipped with a Mettler Toledo TS0801RO sample robot and automated sample carousel. Samples were prepared in 40 μl aluminium pans, the sample lids were automatically pierced by the robot and the analysis undertaken between 30 and 250° C. at 10° C./min. Typically, 1-3 mg of sample was used for analysis and the analysis was performed under dry nitrogen purged at 50 mlmin⁻¹. The instrument was calibrated for energy and temperature using certified indium.

Thermogravimmetric analysis (TGA) analysis was determined using a Mettler Toledo thermogravimetric analyser (TGA851e) equipped with a TS0801 RO sample robot and automated sample carousel. Each pan lid was pierced manually before analysis and run between 30 and 400° C. at 10° C./min. Typically, 1-3 mg of sample was used for analysis. A nitrogen purge at 60 mlmin⁻¹ was maintained over the sample during analysis. The instrument was calibrated for temperature.

Powder X-ray diffraction (PXRD) data were collected on a Bruker AXS C2 GADDS diffractometer using Cu K_{α} radiation (40 kV, 40 mA), an automated XYZ stage, a laser video microscope for auto-sample positioning and a HiStar 2 dimensional area detector. The X-ray optics consisted of a single Göbel multilayer mirror coupled with a pinhole collimator of 0.3 mm. The beam divergence, i.e. the effective size of the X-ray beam on the sample, was approximately 4 mm. A θ-θ continuous scan mode was employed with a sample to detector distance which gave an effective 20 range of 3.2° to 42.7°. Typically the sample was exposed to the X-ray beam for 120 seconds. Samples were prepared as flat plate specimens using material as received without grinding. Approximately 1-2 mg of the sample was lightly pressed on a glass slide to obtain a flat surface.

Dynamic vapour sorption (DVS) analysis was performed on a Surface Measurement systems (SMS) DVS-Intrinsic moisture sorption analyser. The instrument was controlled by SMS Analysis Suite software (DVS-Intrinsic Control v1.0.0.30). Analysis of the data was performed using Microsoft Excel 2007 together DVS Standard Analysis Suite (v6.0.0.7). Sample temperature was maintained at 25° C. and the sample humidity was obtained by mixing streams of wet and dry nitrogen at a total flow rate of 200 mlmin⁻¹. The relative humidity was measured using a calibrated Rotronic probe (dynamic range 1-100% Relative Humidity (RH)) located close to the sample. The weight change of the sample as a function of % RH was constantly monitored by the microbalance (accuracy±0.005 mg). Typically a PXRD would be run prior to analysis. 20 mg of sample was then placed in a tared stainless steel mesh basket under ambient conditions. The sample was loaded and unloaded at 40% RH and 25° C. (typical room conditions) and the sample subjected to a graduated DVS regime over 2 cycles using the parameters shown in Table 1. A DVS isotherm was calculated from this data and a final PXRD was performed after analysis to check for change in solid state form.

### Table 1. Method Parameters for DVS Experiment

TABLE 1
Method parameters for DVS experiment

| Parameter | Setting |
|---|---|
| Sorption - cycle 1 (% RH) | 40-90 |
| Desorption - cycle 1 (% RH) | 90-0 |
| Sorption - cycle 2 (% RH) | 0-90 |
| Desorption - cycle 2 (% RH) | 90-0 |
| Sorption - cycle 3 (% RH) | 0-40 |
| Intervals (% RH) | 10 |
| dmdt (% min⁻¹) | 0.002 |
| Sample temperature (° C.) | 25 |

### For the Analysis of the Crystalline Forms of Examples 3, 4, 5, 7 and 8:

X-Ray Powder Diffraction (XRPD)—PANalytical X'Pert machine in 2Ø-Ø configuration or a PANalytical Cubix machine in Ø-Ø configuration over the scan range 2° to 40° 2Ø with 100-second exposure per 0.02° increment. The X-rays were generated by a copper long-fine focus tube operated at 45 kV and 40 mA. The wavelength of the copper X-rays was 1.5418 Å. The Data was collected on zero background holders on which ˜2 mg of the compound was placed. The holder was made from a single crystal of silicon, which had been cut along a non-diffracting plane and then polished on an optically flat finish. The X-rays incident upon this surface were negated by Bragg extinction.

Differential Scanning calorimetry (DSC) thermograms were measured using a TA Q1000 Differential Scanning calorimeter, with aluminium pans and pierced lids. The sample weights varied between 0.5 to 5 mg. The procedure was carried out under a flow of nitrogen gas (50 ml/min) and the temperature studied from 25 to 300° C. at a constant rate of temperature increase of 10° C. per minute.

Thermogravimetric Vapour Sorption (TGA) thermograms were measured using a TA Q500 Thermogravimetric Analyser, with platinum pans. The sample weights varied between 1 and 5 mg. The procedure was carried out under a flow of nitrogen gas (60 ml/min) and the temperature studied from Room Temperature to 300° C. at a constant rate of temperature increase of 10° C. per minute.

Gravimetric Vapour Sorption (GVS) profiles were measured using a Surface Measurements Systems Dynamic Vapour Sorption DVS-1 or a DVS Advantage instrument. The solid sample ca. 1-5 mg was placed into a glass vessel and the weight of the sample was recorded during a dual cycle step method (40 to 90 to 0 to 90 to 0% relative humidity (RH), in steps of 10% RH).

### Intermediate 1

### (R)-3-(3-Fluoro-phenoxy)-1-aza-bicyclo[2.2.2]octane

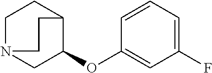

A solution of (R)-1-aza-bicyclo[2.2.2]octan-3-ol (1.25 g), CuI (93.1 mg), 1,10-phenanthroline (176 mg), Cs₂CO₃ (3.19 g) and 3-fluoro-iodo-benzene (1.11 g) in toluene (2.5 mL) was heated at 100° C. for 20 h. The reaction mixture was cooled, diluted with ethyl acetate and filtered through Celite. The insoluble material was washed several times with ethyl acetate. The filtrate was washed with 5% copper sulphate solution, water, dried (MgSO₄), filtered and evaporated in vacuo. Purification by SCX gave (R)-3-(3-fluoro-phenoxy)-1-aza-bicyclo[2.2.2]octane (490 mg, 45%) as a brown oil. LCMS (Method 2, Rt 2.09 min). MH⁺=222.

Intermediates 2-3 were prepared from (R)-1-aza-bicyclo[2.2.2]octan-3-ol and the appropriate aryl iodide by analogy with the procedure described for Intermediate 1. Data for Intermediates 2-3:

| Intermediate | | LCMS (Method, |
|---|---|---|
| Number | Structure | Retention Time, MH+) |
| 2 | 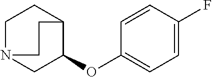 | 2, 2.05 min, 222 |
| 3 | 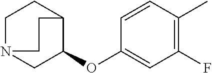 | 2, 2.21 min, 236 |

### Intermediate 4

### (R)-3-(3-Fluoro-phenylsulfanyl)-1-aza-bicyclo[2.2.2]octane

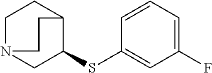

(R)-3-(3-Fluoro-phenylsulfanyl)-1-aza-bicyclo[2.2.2]octane was prepared from 3-fluorothiophenol as follows: A solution of 3-fluorothiophenol (5 g) in DMF (5 ml) was added slowly to a suspension of NaH (1.56 g of 60% dispersion in mineral oil) in DMF (40 mL) at room temperature. After 30 min, a solution of methanesulfonic acid (S)-(1-aza-bicyclo[2.2.2]oct-3-yl)ester (5.3 g) (J. Med. Chem., 1992, 35, 2392-2406) in DMF (5 mL) was added to the mixture dropwise and the reaction mixture was heated at 70° C. overnight. The reaction mixture was partitioned between ethyl acetate and 1 N NaOH solution. The layers were separated and the aqueous phase was extracted with ethyl acetate. The combined organic layers were washed with brine, dried (MgSO₄), filtered and evaporated in vacuo. Purification by SCX chromatography gave (R)-3-(3-fluoro-phenylsulfanyl)-1-aza-bicyclo[2.2.2]octane (4.5 g, 73%). Data for Intermediate 4: NMR (300 MHz, MeOD): 7.33 (1H, td, J=8.04, 6.01 Hz), 7.22-7.13 (2H, m), 7.01-6.93 (1H, m), 3.81-3.70 (1H, m), 3.58-3.48 (1H, m), 3.14-2.91 (2H, m), 2.92-2.77 (2H, m), 2.26-2.15 (1H, m), 2.01-1.77 (4H, m), 1.70-1.58 (1H, m).

### Intermediate A

### (R)-(5-Chloromethyl-isoxazol-3-yl)-cyclohexyl-phenyl-methanol

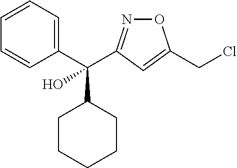

The title compound was obtained from (R)-cyclohexyl-hydroxy-phenyl-acetic acid as follows:

Step 1: 1,1′-Carbonyl diimidazole (25.0 g, 154 mmol) was added to a stirred suspension of (R)-cyclohexyl-hydroxy-phenyl-acetic acid (30.0 g, 128 mmol) in dry THF (600 mL). After stirring for 90 mins at room temperature, sodium borohydride (11.6 g, 307 mmol) was added portionwise over a period of 1 hour. The reaction mixture was then left to stir at room temperature overnight. The reaction was quenched by the addition of water (100 mL) then extracted with DCM. The combined organic phases were dried (MgSO₄), filtered and evaporated in vacuo to give a crude solid. Purification by silica gel chromatography (eluting with 0-5% methanol in DCM) gave (R)-1-cyclohexyl-1-phenyl-ethane-1,2-diol (20.7 g, 73%). ¹H NMR (400 MHz, CDCl₃): δ 7.41-7.33 (4H, m), 7.28-7.24 (1H, m), 3.99 (1H, d), 3.83 (1H, d), 2.68 (1H, br s), 1.86-1.80 (1H, m), 1.78-1.64 (3H, m), 1.63-1.57 (1H, m), 1.47-1.41 (1H, m), 1.27-0.94 (5H, m).

Step 2: A solution of oxalyl chloride (15.5 mL, 201 mmol) in dry DCM (900 mL) was cooled to −78° C. under a nitrogen atmosphere. A solution of DMSO (28.5 mL, 401 mmol) in DCM (25 mL) was added dropwise then the mixture stirred at −78° C. for 10 mins. A solution of (R)-1-cyclohexyl-1-phenyl-ethane-1,2-diol (29.5 g, 134 mmol) in DCM (250 mL) was added dropwise over the course of 1 hour giving a thick slurry. The internal temperature was allowed to reach −45° C. Triethylamine (92.8 mL, 669 mmol) was added dropwise and after complete addition the mixture was allowed to warm to room temperature. The mixture was washed with 1 N hydrochloric acid (500 mL×2), water (500 mL) and brine (500 mL) then dried (MgSO₄), filtered and evaporated to give an orange-coloured oil. This was dissolved in IMS (320 mL) and added portionwise to a preformed solution of hydroxylamine hydrochloride (14.0 g, 201 mmol) and sodium carbonate (21.3 g, 201 mmol) in water (210 mL). The resulting emulsion was stirred at room temperature overnight then partitioned between DCM and water. The organic layer was washed with water and brine, then dried (MgSO₄), filtered and evaporated in vacuo. Purification by silica gel chromatography (eluting with 0-15% EtOAc in cyclohexane) gave (R)-cyclohexyl-hydroxy-phenyl-acetaldehyde oxime (25.9 g, 83%). ¹H NMR (400 MHz, CDCl₃): δ 7.76 (1H, s), 7.44-7.41 (2H, m), 7.37-7.33 (2H, m), 7.27-7.23 (1H, m), 7.22 (1H, br s), 3.34 (1H, s), 1.90-1.60 (5H, m), 1.37-1.05 (6H, m).

Step 3: A solution of (R)-cyclohexyl-hydroxy-phenyl-acetaldehyde oxime (8 g, 34 mmol) and 2,6-lutidine (10 mL, 86 mmol) in DCM (150 mL) was cooled in an ice-bath. Trimethylsilyl trifluoromethanesulfonate (15.6 mL, 86 mmol) was added dropwise. The mixture was stirred for 10 minutes at 0° C. then allowed to warm to room temperature for 30 mins. The reaction was quenched by addition of water (50 mL). The organic phase was isolated by passage though a phase separation cartridge and evaporated in vacuo. Purification by silica gel chromatography (eluting with 10-20% EtOAc in cyclohexane) gave a mixture of mono and bis TMS-protected compounds. This was dissolved in methanol and left at room temperature overnight and evaporated in vacuo to give (R)-cyclohexyl-phenyl-trimethylsilanyloxy-acetaldehyde oxime (10 g, 96%). ¹H NMR (400 MHz, CDCl₃): δ 7.62 (1H, s), 7.32-7.28 (4H, m), 7.26-7.21 (1H, m), 7.11 (1H, s), 1.93-1.85 (2H, m), 1.76-1.71 (1H, m), 1.68-1.56 (2H, m), 1.49-1.42 (1H, m), 1.27-0.78 (5H, m), 0.11 (9H, m).

Step 4: A solution of (R)-cyclohexyl-phenyl-trimethylsilanyloxy-acetaldehyde oxime (6 g, 19.6 mmol) was formed in dry DCM (400 mL) and cooled to −78° C. Under reduced lighting, a solution of tert-butylhypochlorite (4.3 g, 39.3 mmol) in DCM (10 mL) was added dropwise. After 2 hours at −78° C. a solution of triethylamine (4.1 mL, 29.4 mmol) in DCM (10 mL) was added dropwise. After a further 10 mins at −78° C. the mixture was allowed to warm to 0° C. At this point, propargyl chloride (14.4 mL, 196 mmol) was added and the mixture was allowed to warm to room temperature overnight. The mixture was washed with brine (200 mL), dried (Na₂SO₄), filtered and evaporated. Purification by silica gel chromatography (eluting with 0-10% EtOAc in cyclohexane) gave crude 5-chloromethyl-3-((R)-cyclohexyl-phenyl-trimethylsilanyloxy-methyl)-isoxazole. This was re-dissolved in THF (100 mL), cooled in an ice-bath and a solution of tetrabutylammonium fluoride (19.6 mL of 1 M in THF) was added dropwise. This mixture was stirred for 30 mins at 0° C. then partitioned between ethyl acetate and water. The organic phase was dried (Na₂SO₄), filtered and evaporated in vacuo. Purification by silica gel chromatography (eluting with 0-20% EtOAc in cyclohexane) gave the title compound as a white solid (3.5 g, 58%). ¹H NMR (400 MHz, CDCl₃): δ 7.51 (2H, m), 7.32 (2H, m), 7.25-7.21 (1H, m), 6.29 (1H, s), 4.52 (2H, s), 2.80 (1H, s), 2.34-2.28 (1H, m), 1.81-1.76 (1H, m), 1.72-1.62 (3H, m), 1.36-1.02 (6H, m).

### Intermediate B

### (R)-(5-Bromomethyl-[1,3,4]oxadiazol-2-yl)-cyclohexyl-phenyl-methanol

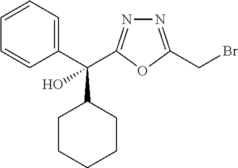

### Step 1

### (R)-Cyclohexyl-hydroxy-phenyl-acetic acid hydrazide

A solution of (R)-cyclohexylmandelic acid (2.34 g) was dissolved in DCM (20 mL), treated with 1,1′-carbonyldiimidazole (1.95 g) and stirred at room temperature for 1 h. The reaction mixture was treated with hydrazine monohydrate (1.0 mL) and stirred for a further 30 minutes. The reaction mixture was diluted with DCM, washed with 1 N NaOH solution and brine, dried (MgSO₄), filtered and evaporated in vacuo to give the title compound as a white solid (2.0 g, 81%). LCMS (Method 2, 2.73 min). MH⁺=249.

### Step 2

### Chloro-acetic acid N′-((R)-2-cyclohexyl-2-hydroxy-2-phenyl-acetyl)-hydrazide

A solution of the foregoing compound (1.0 g) was dissolved in DCM (20 mL) and treated at 0° C. with diisopropylethylamine (0.83 mL) and chloroacetyl chloride (0.39 ml). After warming to room temperature and stirring for 10 minutes, the reaction mixture was diluted with DCM, washed with water and brine, dried (MgSO₄), filtered and evaporated in vacuo to give the desired compound (1.1 g, 73%) as a white solid. LCMS (Method 2, 3.20 min). MH⁺=325.

### Step 3

### (R)-(5-Chloromethyl-[1,3,4]oxadiazol-2-yl)-cyclohexyl-phenyl-methanol

A solution of the foregoing compound (170 mg), tosyl chloride (96 mg) and 1,2,2,6,6-pentamethylpiperidine (175 mg) in DCM (2 mL) was stirred at room temperature overnight. The reaction mixture was diluted with DCM, washed with NaHCO₃ solution (twice), brine, dried (MgSO₄), filtered and evaporated in vacuo. Purification by column chromatography (silica, 0-100% cyclohexane/ethyl acetate) gave the title compound as a white solid (105 mg, 63%). Data for the title compound: LCMS (Method 2, 3.79 min). MH⁺=307.

Step 4: A solution of the foregoing compound (4.66 g) and lithium bromide (6.6 g) in acetone (200 ml) was refluxed overnight. The reaction mixture was cooled, evaporated in vacuo and partitioned between water and ethyl acetate. The organic phase was separated, dried (MgSO₄), filtered and evaporated in vacuo. The resulting solid was redissolved in acetone (200 ml), treated with lithium bromide (6.6 g) and heated to reflux overnight. The reaction mixture was cooled, concentrated in vacuo and partitioned between water and ethyl acetate. The organic phase was separated, dried (MgSO₄), filtered and evaporated in vacuo to yield the title compound 4.65 g, 84%). Data for the title compound: LCMS (Method 2, 3.90 min). MH⁺=353. ¹H NMR δ (ppm) (CHCl₃−d): 7.60-7.53 (2H, m), 7.41-7.25 (3H, m), 4.49 (2H, s), 3.28 (1H, s), 2.33 (1H, s), 1.85-1.73 (1H, m), 1.68 (3H, s), 1.44-1.09 (6H, m).

### Intermediate C

### (5-Bromomethyl-isoxazol-3-yl)-diphenyl-methanol

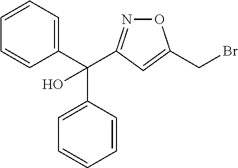

The title compound was obtained from methyl 5-methylisoxazole-3-carboxylate as follows:

Step 1. Phenylmagnesium Bromide (3 M solution in ether; 100 mL) was added dropwise to a solution of methyl 5-methylisoxazole-3-carboxylate (20.2 g) in anhydrous THF (300 mL) at −10° C. under a nitrogen atmosphere. The reaction mixture was stirred at −10° C. for 5 mins, then allowed to warm up to RT and left to stand for 18 hours. The reaction mixture was poured into cold 1 M HCl (300 mL) and extracted with ether. The combined organic extracts were washed with NaHCO₃, water, and brine, dried (MgSO₄), filtered and evaporated in vacuo to give (5-methyl-isoxazol-3-yl)-diphenyl-methanol (37.21 g, 98%) as a waxy solid. ¹H NMR (400 MHz, CDCl₃): δ 7.39-7.25 (m, 10H), 5.84 (s, 1H), 3.69 (s, 1 H), 2.38 (s, 3H).

Step 2. Dry 1,2-DCE (500 mL) was purged with argon for 15 mins. (5-Methyl-isoxazol-3-yl)-diphenyl-methanol (37.9 g) was added under nitrogen with stirring followed by NBS (28.0 g) and AlBN (4.7 g). The reaction mixture was stirred at 80° C. for 1 hour. Further NBS (28.0 g) and AlBN (4.7 g) was added to the reaction mixture and stirring continued at 80° C. for 3 hours. The reaction mixture was allowed to cool to RT, poured into 1M HCl (500 mL) and extracted with ether. The combined organic extracts were washed with NaHCO₃, water, and brine, dried (MgSO₄), filtered and evaporated in vacuo. Purification by silica gel chromatography eluting with 10-100% cyclohexane-DCM gave the title compound (26.0 g, 52%) as a pale yellow solid containing smaller amounts of unchanged starting material, and dibrominated- and tribrominated impurities. Data for the title compound: ¹H NMR (400 MHz, CDCl₃): δ 7.38-7.23 (m, 10H), 6.18 (s, 1H), 4.35 (s, 2H), 3.63 (s, 1H).

The title compound may also be obtained from ethyl 2-chloro-2-(hydroxyimino)acetate as follows:

### Step A

### 5-Hydroxymethyl-isoxazol-3-yl)-diphenyl-methanol

A solution of triethylamine (69 mL) in ether (31 mL) was added slowly over 4 h with the aid of a syringe pump to a briskly stirred solution of propargyl alcohol (37.5 mL) and ethyl 2-chloro-2-(hydroxyimino)acetate (75 g) in ether (500 mL) at room temperature. The reaction mixture was then allowed to stir overnight, filtered and the filtrate washed with water (twice). The aqueous phases were combined, saturated with sodium chloride and re-extracted with ethyl acetate (twice). The combined organic phases were dried (MgSO₄), filtered and evaporated in vacuo to give a thick oil (82 g) comprised mainly of 5-hydroxymethyl-isoxazole-3-carboxylic acid ethyl ester. This was dissolved in THF (700 mL), cooled to −10° C. and treated with a solution of phenylmagnesium chloride (750 mL, 2.0 M in THF) keeping the temperature below −2° C. The reaction mixture was warmed to room temperature and stirred for 1 h. The reaction mixture was poured carefully into ice-cold concentrated hydrochloric acid (200 mL) and ice (500 mL) and the layers separated. The aqueous layer was extracted with ether. The combined organic layers were washed with brine, dried, filtered and evaporated in vacuo. Trituration with ether gave (5-hydroxymethyl-isoxazol-3-yl)-diphenyl-methanol (82.3 g, 59% (2 steps)) as a white solid.

¹H NMR (300 MHz, DMSO): δ 7.39-7.25 (m, 10H), 6.82 (s, 1H), 6.34 (s, 1H), 5.62 (t, J=6.0 Hz, 1H), 4.54 (d, J=6.0 Hz, 2H).

### Step B

### (5-Bromomethyl-isoxazol-3-yl)-diphenyl-methanol

A solution of the foregoing compound (40 g) and tetrabromomethane (70.8 g) in DCM (350 mL) was cooled to −15° C. and treated portionwise with triphenylphosphine (48.5 g), keeping the temperature below −8° C. The reaction was allowed to warm to 10° C., then poured directly onto a silica gel pad and eluted with DCM (2500 mL). The eluent was evaporated and purified by column chromatography (0-25% ethyl acetate in cyclohexane) to give the title compound (43 g, 88%) as a thick, straw-coloured oil. ¹H NMR (400 MHz, CDCl₃): δ 7.38-7.23 (m, 10H), 6.18 (s, 1H), 4.35 (s, 2H), 3.63 (s, 1H).

### EXAMPLE 1

### (R)-1-[3-((R)-Cyclohexyl-hydroxy-phenyl-methyl)-isoxazol-5-ylmethyl]-3-(4-fluoro-phenoxy)-1-azonia-bicyclo[2.2.2]octane chloride

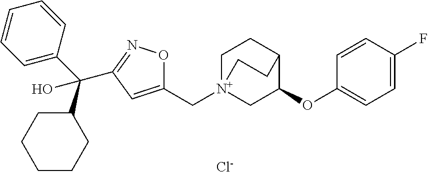

(R)-(5-Chloromethyl-isoxazol-3-yl)cyclohexyl-phenyl-methanol (Intermediate A) (1.74 g) and (R)-3-(4-fluoro-phenoxy)-1-aza-bicyclo[2.2.2]octane (Intermediate 2) (1.26 g) were mixed in acetonitrile (25 mL) and heated at 50° C. for 1 h. The resulting white solid was collected by filtration, washed with ethyl acetate and ether and dried in vacuo to yield the title compound (2.9 g). This was dissolved in boiling acetonitrile (125 ml) and allowed to cool slowly to room temperature whilst being stirred. The resulting crystals were collected by filtration and dried in vacuo to yield the title compound (2.4 g, 81%). Data for Example 1: ¹H NMR (400 MHz, DMSO-d6): δ 7.51-7.46 (m, 2H), 7.32 (t, 2H), 7.25-7.12 (m, 3H), 7.02-6.95 (m, 2H), 6.79 (s, 1H), 5.90 (s, 1H), 4.88 (s, 1H), 4.77 (s, 2H), 3.91 (dd, 1H), 3.54-3.34 (m, 5H), 2.39 (s, 1H), 2.24-2.09 (m, 2H), 2.06-1.97 (m, 1H), 1.94-1.80 (m, 2H), 1.68 (d, 1H), 1.58 (d, 3H), 1.28-1.13 (m, 3H), 1.10-0.98 (m, 3H). LCMS (Method 1, 8.68 min). M⁺=491.

### EXAMPLE 2

### (R)-1-[3-((R)-Cyclohexyl-hydroxy-phenyl-methyl)-isoxazol-5-ylmethyl]-3-(4-fluoro-phenoxy)-1-azonia-bicyclo[2.2.2]octane benzenesulfonate

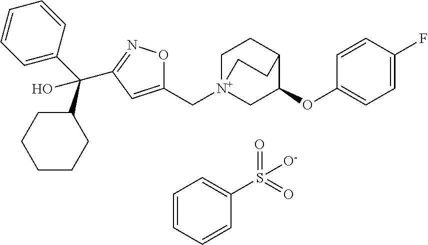

A solution of (R)-1-[3-((R)-cyclohexyl-hydroxy-phenyl-methyl)-isoxazol-5-ylmethyl]-3-(4-fluoro-phenoxy)-1-azonia-bicyclo[2.2.2]octane; chloride (Example 1) (2.0 g) was dissolved in DCM (20 ml) and stirred briskly with a solution of sodium benzenesulfonate (3.4 g) in water (20 ml). The organic layer was separated and stirred briskly again with a solution of sodium benzenesulfonate (3.4 g) in water (20 ml). The organic layer was dried (MgSO₄), filtered and evaporated in vacuo to give the title compound as a white foam. This was dissolved in boiling propan-2-ol (48 ml). The hot solution was filtered, and the filtrate was allowed to cool slowly to room temperature while being stirred. After 2 h, the mixture was cooled to 0° C., and the crystals were collected by filtration and dried in vacuo. The title compound (2.1 g) was obtained in 85% yield. ¹H NMR δ (ppm) (DMSO-d₆): 7.62-7.58 (2 H, m), 7.52-7.47 (2H, m), 7.35-7.26 (5H, m), 7.26-7.13 (3H, m), 7.02-6.95 (2H, m), 6.80 (1H, s), 5.89 (1H, s), 4.88 (1H, s), 4.75 (2H, s), 3.91 (1H, dd, J=13.17, 8.11 Hz), 3.58-3.35 (5H, m), 2.40 (1H, s), 2.25-1.95 (3H, m), 1.96-1.80 (2H, m), 1.69 (1H, d, J=10.55 Hz), 1.63-1.52 (3H, m), 1.29-0.96 (6H, m). LCMS (Method 1, 8.73 min). M⁺=491.

A sample of crystalline material was analysed by DSC, TGA, PXRD and DVS.

The melting temperature was determined by DSC at 10° C./min and found to have a sharp endothermic event with an onset temperature of 178° C. (±1° C.). Weight loss prior to melting was negligible by TGA. PXRD analysis showed the sample to be highly crystalline (see FIG. 1). DVS analysis produced a weight increase of 0.2% (% w/w) at 80% RH (±0.1%).

### EXAMPLE 3

### (R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane chloride

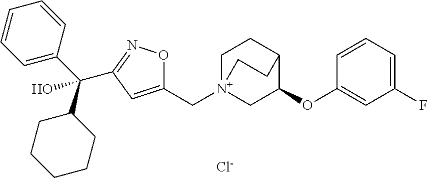

(R)-(5-Chloromethyl-isoxazol-3-yl)cyclohexyl-phenyl-methanol (Intermediate A) (3.00 g) and (R)-3-(3-fluoro-phenoxy)-1-aza-bicyclo[2.2.2]octane (Intermediate 1) (2.17 g) were mixed in acetonitrile (60 mL) and heated at 50° C. for 2 h. The reaction mixture was evaporated in vacuo and purified by silica gel chromatography (eluting with 1-15% methanol in DCM) to give the title compound as a white foam. This was dissolved in boiling acetonitrile (500 ml) and allowed to cool slowly to room temperature. The resulting white crystals were collected by filtration and dried in vacuo to give the title compound (3.9 g, 75%). ¹H NMR (400 MHz, DMSO-d6): δ 7.49 (dd, 2H), 7.40-7.29 (m, 3H), 7.25-7.20 (m, 1H), 6.93-6.79 (m, 4H), 5.90 (s, 1H), 4.96 (s, 1H), 4.77 (s, 2H), 3.95 (dd, 1H), 3.49 (d, 4H), 2.43 (s, 1H), 2.26-2.10 (m, 2H), 2.07-1.98 (m, 1H), 1.95-1.82 (m, 2 H), 1.69 (d, 1H), 1.59 (s, 4H), 1.28-1.14 (m, 3H), 1.10-0.98 (m, 3H). LCMS (Method 1, 8.70 min). M⁺=491.

A sample of crystalline material was analysed by DSC, XRPD and GVS.

The melting temperature was determined by DSC and found to have a broad endothermic event (melt) onset approximately 134° C. (±2° C.). XRPD analysis showed the sample to be crystalline (see FIG. 2). GVS analysis produced a mass increase of approximately 5% 1^{st} cycle and 6.5% 2^{nd} cycle at 80% RH.

### EXAMPLE 4

### (R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate

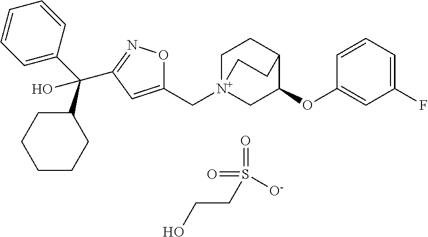

A solution of (R)-1-[3-((R)-cyclohexyl-hydroxy-phenyl-methyl)-isoxazol-5-ylmethyl]-3-(3-fluoro-phenoxy)-1-azonia-bicyclo[2.2.2]octane; chloride (Example 3) (3.2 g) in warm DCM (50 ml) and methanol (0.5 ml) was stirred briskly and treated with a solution of ammonium isethionate (5 g) in water (20 ml). The reaction mixture was stirred at room temperature for 1 h, then cooled to 0° C. and stirred for 0.5 h. The resulting white precipitate was collected by filtration and washed with water and ether and dried in vacuo. The precipitate was dissolved in boiling acetonitrile (172 ml). The resulting solution was filtered whilst hot, and allowed to cool slowly to room temperature whilst being stirred. After 2 h, the resulting white crystals were collected by filtration and dried in vacuo to give the title compound (3.07 g, 82%). ¹H NMR δ (ppm) (DMSO-d₆): 7.47-7.42 (2H, m), 7.35-7.25 (3H, m), 7.21-7.13 (1H, m), 6.81 (4H, d, J=43.75 Hz), 5.84 (1H, s), 4.92 (1H, s), 4.70 (2H, s), 4.40 (1H, t, J=5.72 Hz), 3.90 (1H, dd, J=13.18, 8.10 Hz), 3.58 (2H, td, J=6.74, 5.72 Hz), 3.48-3.29 (5H, m), 2.56 (2H, t, J=6.74 Hz), 2.39 (1H, s), 2.21-2.04 (2H, m), 2.03-1.94 (1H, m), 1.93-1.77 (2H, m), 1.64 (1H, d, J=10.36 Hz), 1.54 (3H, d, J=9.07 Hz), 1.24-1.10 (3H, m), 1.10-0.93 (3H, m). LCMS (Method 1, 8.72 min). M⁺=491.

A sample of crystalline material was analysed by DSC, XRPD and GVS.

The melting temperature was determined by DSC and found to have a sharp melt onset at approximately 214° C. (±2° C.). XRPD analysis showed the sample to be crystalline (see FIG. 3). GVS analysis produced no mass increase at 80% RH.

### EXAMPLE 5

### (R)-1-[5-((R)-Cyclohexyl-hydroxy-phenyl-methyl)-[1,3,4]oxadiazol-2-ylmethyl]-3-(4-fluoro-phenoxy)-1-azonia-bicyclo[2.2.2]octane bromide

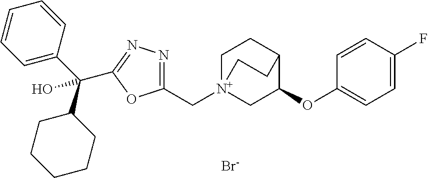

A solution of (R)-(5-bromomethyl-[1,3,4]oxadiazol-2-yl)-cyclohexyl-phenyl-methanol (Intermediate B) (2.93 g) and (R)-3-(4-fluoro-phenoxy)-1-aza-bicyclo[2.2.2]octane (Intermediate 2) (1.8 g) in acetonitrile (60 ml) was heated at 50° C. overnight. The reaction mixture was evaporated in vacuo and triturated with ether to yield the title compound (4.7 g), which was recrystallized from boiling ethyl acetate. ¹H NMR (400 MHz, DMSO-d6): δ 7.44-7.39 (m, 2H), 7.34-7.21 (m, 3H), 7.16-7.09 (m, 2H), 6.97-6.90 (m, 2H), 6.39 (s, 1H), 4.92 (s, 2H), 4.82 (s, 1H), 3.97-3.87 (m, 1H), 3.59-3.37 (m, 5H), 2.38 (s, 1H), 2.22 (t, 1H), 2.11 (s, 1H), 2.00 (s, 1H), 1.84 (s, 2H), 1.66 (s, 2H), 1.57 (t, 2H), 1.32 (d, 1H), 1.23-1.00 (m, 3H), 1.03-0.88 (m, 2H). LCMS (Method 1, 8.29 min). M⁺=492.

A sample of crystalline material was analysed by DSC, XRPD and GVS.

The melting temperature was determined by DSC and a double endothermic event was observed. The melt onset was assumed to be approximately 169° C. (±2° C.). XRPD analysis showed the sample to be crystalline (see FIG. 4). GVS analysis produced a mass increase of approximately 0.8% at 80% RH.

### EXAMPLE 6

### (R)-3-(3-Fluoro-phenylsulfanyl)-1-[3-(hydroxy-diphenyl-methyl)-isoxazol-5-ylmethyl]-1-azonia-bicyclo[2.2.2]octane bromide

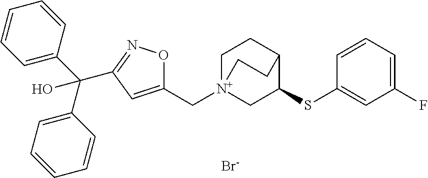

A solution of (5-bromomethyl-isoxazol-3-yl)-diphenyl-methanol (Intermediate C) (1.1 g of an approximately 40% pure sample) and (R)-3-(3-fluoro-phenylsulfanyl)-1-aza-bicyclo[2.2.2]octane (Intermediate 4) (218 mg) in acetonitrile (10 ml) was stirred at room temperature for 1 h. The resulting precipitate was collected by filtration and dried in vacuo. This was dissolved in boiling acetonitrile (130 ml), filtered whilst hot, and allowed to cool slowly to room temperature whilst stirring. The resulting crystals were collected by filtration and dried in vacuo to yield the title compound (312 mg, 51%). ¹H NMR δ (ppm) (400 MHz, CH₃OH-d₄): 7.40-7.22 (13H, m), 7.09-7.03 (1H, m), 6.83 (1H, s), 4.71 (2H, s), 4.07-3.98 (2H, m), 3.69-3.38 (5H, m), 2.50-2.39 (1H, m), 2.29-2.25 (1H, m), 2.24-2.14 (1H, m), 2.18-1.93 (2H, m). LCMS (Method 1, 8.36 min). M⁺=501.19.

### EXAMPLE 7

### (R)-3-(3-Fluoro-4-methyl-phenoxy)-1-[3-(hydroxy-diphenyl-methyl)-isoxazol-5-ylmethyl]-1-azonia-bicyclo[2.2.2]octane bromide

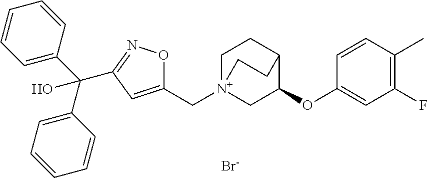

A solution of (5-bromomethyl-isoxazol-3-yl)-diphenyl-methanol (Intermediate C) (4.7 g of an approximately 67% pure sample) and (R)-3-(3-fluoro-4-methyl-phenoxy)-1-aza-bicyclo[2.2.2]octane (Intermediate 3) (2 g) in acetonitrile (50 ml) was heated at 50° C. for 1.5 h. The reaction mixture was cooled and the solid was collected by filtration and washed with ethyl acetate and ether and dried in vacuo to give the title compound (4.36 g, 88%). This was dissolved in boiling propan-2-ol (760 ml), filtered whilst hot, and allowed to cool slowly to room temperature whilst stirring. The resulting crystals were collected by filtration and dried in vacuo to yield the title compound (3.72 g). ¹H NMR δ (ppm) (400 MHz, CH₃OH-d₄): 7.39-7.26 (10H, m), 7.16 (1H, t, J=8.63 Hz), 6.84 (1H, s), 6.75-6.66 (2H, m), 4.93-4.87 (1H, m), 4.79-4.70 (2H, m), 4.03-3.95 (1H, m), 3.67-3.48 (5H, m), 2.56-2.52 (1H, m), 2.40-2.31 (1H, m), 2.20-2.11 (4H, m), 2.10-1.93 (2H, m). LCMS (Method 1, 8.37 min). M⁺=499.20.

A sample of crystalline material was analysed by DSC, XRPD and GVS.

The melting temperature was determined by DSC and found to have a sharp melt onset at approximately 242° C. (±2° C.). XRPD analysis showed the sample to be crystalline (see FIG. 5). GVS analysis produced a mass increase of approximately 0.1% at 80% RH.

### EXAMPLE 8

### (R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate

To a stirred suspension of (R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane chloride¹ (155.83 g) and DCM (2380 mL) in a 5 L flask equipped with an overhead stirrer was added MeOH (23.8 mL) in one portion. After stirring for a few minutes a solution formed. To the stirred solution of the chloride salt was added a solution of isethionic acid, ammonium salt (61.60 g) in water (945 mL) over 5 minutes. The resulting two-phase reaction mixture was stirred vigorously and after a few minutes some seed crystals of (R)-1-[3-((R)-cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate were added. A few more were added after a further 35 minutes of stirring. Traces of solid formation were observed around the sides of the flask. It was stirred at room temperature for a further 2.5 hours and a dense precipitate began to form. Examination of a small aliquot of the reaction mixture under a microscope showed crystalline material. The stirred reaction mixture was cooled in an ice bath (with internal temperature 4° C. for 35 minutes). The solid became more granular. The solid was collected by filtration and washed with cold water (total volume 3.1 L in 400-60 mL portions) and then with ether (5×500 mL). It was sucked dry in air and then dried in vacuo at 40° C. overnight and then for a further 6 hours to give the product as a white crystalline solid (152.48 g). LC-MS (Method 2): Rₜ 8.91 min, m/z 491 [M]⁺. Purity>99%.

The product (152.48 g) was then dissolved with stirring in refluxing IMS (2.8 L) and the hot solution was filtered. This solution was kept hot and stirred in a 10 L heated jacket reactor whilst the remaining material (151.64 g) was dissolved in refluxing IMS (2.8 L) and then filtered hot. The two solutions were combined in a 10 L heated jacket reactor and stirred and refluxed. A small amount of material had started to crystallise out, so further IMS (350 mL) was added until a solution formed. The stirred solution (stirring speed 88-89 rpm) was gradually allowed to cool [78° C. (reflux temperature) to 76.5° C. (internal temperature) over about 1 h and then 76.5-20° C. (internal temperature) over 4.5 hours and then stirred at 20° C. overnight]. Seed crystals were added to the stirred solution at 77° C., 69° C. and 59° C. Solid material had begun to crystallise out at base of reactor. More crystallisation was observed over the next few minutes as the mixture cooled down further. After stirring overnight the solid was collected by filtration, washed with cold IMS (˜300 ml) and dried by suction in air (for 2.5 hours) and then in vacuo at 40° C. overnight to give crystalline (R)-1-[3-((R)-cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate (274.48 g). LC-MS (Method 2): Rₜ 8.84 min, m/z 491 [M]⁺. Purity>99%. ¹A preparation of (R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane chloride is described in Example 3 and in WO 2008/099186.

A sample of crystalline material was analysed by XRPD, GVS and DSC. The melting temperature as determined by DSC was found to be 213° C. (onset) (±2° C.). GVS determination gave a weight increase of 0.15% at 80% RH (±0.3%). An XRPD spectrum is presented in FIG. 6.

### Biological Activity of Muscarinic Antagonists

The inhibitory effects of compounds of the muscarinic antagonists were determined by a Muscarinic Receptor Radioligand Binding Assay.

Recombinant human M3 receptor was expressed in CHO-K1 cells. Cell membranes were prepared and binding of [3H]-N-methyl scopolamine ([3H]-NMS) and compounds was assessed by a scintillation proximity assay (SPA). The incubation time was 16 hours at ambient temperature in the presence of 1% (v/v) DMSO. The assay was performed in white 96 well clear-bottomed NBS plates (Corning). Prior to the assay, the CHO cell membranes containing M3 receptor were coated onto SPA WGA (Wheat germ agglutinin) beads (GE Healthcare). Non specific binding was determined in the presence of 1 μM Atropine.

Radioactivity was measured on a Microbeta scintillation counter (PerkinElmer) using a 3H protocol with a 2 minutes per well read time. Compound inhibition of [3H]-NMS binding was determined typically using concentrations in the range 0.03 nM to 1 μM and expressed as percent inhibition relative to the plate specific radioligand binding for the plate. Concentration dependent inhibition of [3H]-NMS binding by compounds was expressed as pIC50.

All compounds tested exhibited potencies (as Ki values) in the M3 binding assay of less than 5 nM. In particular, Example 1 exhibited a Ki of 0.80 nM, Example 3 exhibited a Ki of 0.66 nM, Example 5 exhibited a Ki of 0.70 nM, Example 6 exhibited a Ki of 0.15 nM and Example 7 exhibited a Ki value of 0.40 nM in the M3 binding assay.

### Preparation of β₂-Adrenoceptor Agonists

The following β₂-adrenoceptor agonists that may be employed in the combination of the present invention may be prepared as follows.

### General Experimental Details for Preparation of β₂-Adrenoceptor Agonists

¹H NMR spectra were recorded on a Varian Inova 400 MHz or a Varian Mercury-VX 300 MHz instrument. The central peaks of chloroform-d (δ_{H} 7.27 ppm), dimethylsulfoxide-d₆ (δ_{H} 2.50 ppm), acetonitrile-d₃ (δ_{H} 1.95 ppm) or methanol-d₄ (δ_{H} 3.31 ppm) were used as internal references. Column chromatography was carried out using silica gel (0.040-0.063 mm, Merck). Unless stated otherwise, starting materials were commercially available. All solvents and commercial reagents were of laboratory grade and were used as received.

The following method was used for LC/MS analysis:

Instrument Agilent 1100; Column Waters Symmetry 2.1×30 mm; Mass APCI; Flow rate 0.7 ml/min; Wavelength 254 nm; Solvent A: water+0.1% TFA; Solvent B: acetonitrile+0.1% TFA; Gradient 15-95%/B 8 min, 95% B 1 min.

Analytical chromatography was run on a Symmetry C₁₈-column, 2.1×30 mm with 3.5 μm particle size, with acetonitrile/water/0.1% trifluoroacetic acid as mobile phase in a gradient from 5% to 95% acetonitrile over 8 minutes at a flow of 0.7 ml/min.

The abbreviations or terms used in the examples have the following meanings:

SCX: Solid phase extraction with a sulfonic acid sorbent
HPLC: High performance liquid chromatography

### DMF: N,N-Dimethylformamide

The β₂-adrenoceptor agonists and the intermediates used in their preparation are herein named, based upon the structures depicted, using the IUPAC NAME, ACD Labs Version 8 naming package.

### β₂-Adrenoceptor Agonist 1

### (BA1): Preparation 1

### N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide dihydrobromide

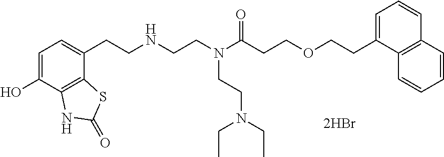

### a) tert-Butyl 3-[2-(1-naphthyl)ethoxy]propanoate

1-Naphthalene ethanol (10 g) was treated with benzyltrimethylammonium hydroxide (Triton B®; 0.9 mL of a 40% solution in methanol) and the resulting mixture stirred in vacuo for 30 minutes. The mixture was then cooled to 0° C. and treated with tert-butyl acrylate (8.19 g). The resulting mixture was slowly warmed to room temperature and stirred overnight. The crude mixture was subsequently absorbed onto aluminium oxide (30 g) and eluted with diethylether (200 mL). The organics were concentrated to give a crude material (16.6 g) which was purified by flash silica chromatography eluting with 1:8, diethylether:hexane to give the subtitled compound (12.83 g).

¹H NMR (CDCl₃) δ 8.05 (dd, 1H), 7.84 (dd, 1H), 7.72 (dd, 1H), 7.54-7.34 (m, 4H), 3.81-3.69 (m, 4H), 3.35 (t, 2H), 2.52-2.47 (m, 2H), 1.45 (s, 9H).

### b) 3-[2-(1-Naphthyl)ethoxy]propanoic acid

tert-Butyl 3-[2-(1-naphthyl)ethoxy]propanoate (6.19 g) was taken up in dichloromethane (30 mL) and treated with trifluoroacetic acid (5 mL). The resulting solution was stirred at room temperature for 2 hours, an additional 1 mL of trifluoroacetic acid was added and the solution stirred overnight. The mixture was concentrated, taken up in 2M sodium hydroxide solution (30 mL) and washed with ether (2×20 mL). The aqueous layer was subsequently acidified (using 1M hydrochloric acid) and extracted with ether (2×30 mL). The combined organics were washed with brine (20 mL), dried over anhydrous magnesium sulphate, filtered and concentrated in vacuo to give the sub-titled compound (5.66 g) as a clear oil.

¹H NMR (CDCl₃) δ 8.05 (bs, 1H), 7.85 (bs, 1H), 7.74 (bs, 1H), 7.50-7.38 (m, 4H), 3.84-3.75 (bm, 4H), 3.39 (bs, 2H), 2.65 (bs, 2H).

### c) N-(2-Diethylaminoethyl)-N-(2-hydroxyethyl)-3-[2-(1-naphthyl)ethoxy]-propanamide

Oxalyl chloride (0.33 g) was added dropwise to a solution of 3-[2-(1-naphthyl)ethoxy]propanoic acid (0.53 g) in dichloromethane (10 mL), dimethylformamide (1 drop) was added and stirring continued at room temperature for 1 hour. The mixture was subsequently concentrated, re-dissolved in dichloromethane (10 mL) and added dropwise to a solution of 2-(2-diethylaminoethylamino)ethanol (0.35 g) and diisopropylethylamine (0.56 g) in dichloromethane (10 mL). The resulting mixture was stirred at room temperature for 1 hour, diluted (dichloromethane, 50 mL), washed with water (2×20 mL), brine (20 mL), dried over magnesium sulfate and concentrated to give the crude product (0.91 g) which was purified by flash column chromatography (eluting with 5-7% methanol in dichloromethane) to give 0.63 g of the sub-titled compound.

¹H NMR (CDCl₃) δ 8.05 (d, 1H), 7.85 (d, 1H), 7.73 (d, 1H), 7.52-7.47 (m, 2H), 7.42-7.35 (m, 2H), 3.84-3.78 (m, 6H), 3.72-3.70 (m, ½H), 3.45-3.35 (m, 6H), 2.79-2.77 (m, 1+½H), 2.62-2.58 (m, 2H), 2.54-2.49 (m, 4H), 1.04-1.01 (m, 6H).

### d) N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide

A solution of dimethylsulfoxide (0.097 g) in dichloromethane (1 mL) was added to a solution of oxalyl chloride (0.079 g) in dichloromethane (10 mL) at −78° C. The reaction was stirred for 15 minutes and then a solution of N-(2-diethylaminoethyl)-N-(2-hydroxyethyl)-3-[2-(1-naphthyl)ethoxy]propanamide (0.22 g) in dichloromethane (1 mL+1 mL wash) was added and the reaction mixture stirred for a further 15 minutes. Triethylamine (0.29 g) was added and the reaction allowed to warm to room temperature over 1 hour, the mixture was subsequently diluted (dichloromethane 30 mL), the organics washed with sodium bicarbonate (20 mL), brine (20 mL), dried over anhydrous magnesium sulphate, filtered and concentrated in vacuo to give the sub-titled compound (0.21 g).

The crude product was dissolved in methanol (10 mL) and 7-(2-aminoethyl)-4-hydroxy-1,3-benthiazol-2(3H)-one hydrochloride (prepared according to the procedure outlined in Organic Process Research & Development 2004, 8(4), 628-642; 0.131 g) was added along with acetic acid (0.1 mL) and water (0.1 mL). After stirring at room temperature for 30 minutes, sodium cyanoborohydride (0.020 g) was added and the reaction mixture was stirred overnight. Ammonia (7N in methanol, 1 mL) was added and the mixture was concentrated. The crude residue was purified by flash column chromatography eluting with 1% ammonia; 5%-7% methanol in dichloromethane. The crude product was used directly in the next step.

### e) N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide dihydrobromide

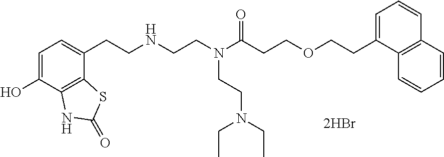

N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide (0.052 g) was dissolved in ethanol (1.5 mL) and treated with 48% hydrobromic acid (21 μl). The white solid dihydrobromide salt (0.058 g) was collected by filtration.

MS: APCI (+ve) 579 (M+1).

¹H NMR δ(DMSO) 11.78-11.71 (m, 1H), 10.11-10.06 (m, 1H), 9.51-9.43 (m, 0.33H), 9.21-9.13 (m, 0.66H), 8.75-8.66 (m, 1H), 8.59-8.51 (m, 1H), 8.06 (d, 1H), 7.95-7.90 (m, 1H), 7.79 (d, 1H), 7.60-7.48 (m, 2H), 7.47-7.39 (m, 2H), 6.87 (t, 1H), 6.76 (dd, 1H), 3.78-3.53 (m, 10H), 3.25-3.09 (m, 10H), 2.91-2.80 (m, 2H), 2.73-2.61 (m, 2H), 1.26-1.15 (m, 6H). NMR indicates approximately 2:1 mixture of rotamers at 298K.

### β₂-Adrenoceptor Agonist 1

### (BA1): Preparation 2

### N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide dihydrobromide

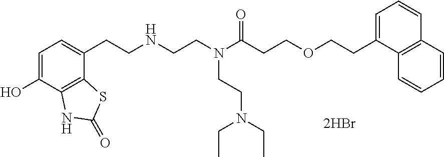

### a) N′-(2,2-Dimethoxyethyl)-N,N-diethyl-ethane-1,2-diamine

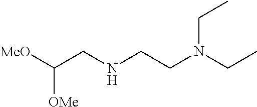

A solution of N,N-diethyl-ethylenediamine (150 g) in methanol (500 mL) was treated dropwise rapidly with glyoxal dimethylacetal (60 wt % soln. in water, 225 g) at 10-15° C. After the addition was complete the solution was warmed to 15° C., then to 22° C. and left at this temperature for 16 hours. The reaction mixture was treated with 5% palladium on carbon (Johnson-Matthey type 38H paste, 15 g) and hydrogenated at 6 bar until the reaction was complete as judged by GC/MS. The catalyst was removed by filtration and the filtrate evaporated to dryness (toluene azeotrope, 2.5 L), affording 196.2 g of the sub-titled compound.

¹H NMR (CDCl₃): 4.48 (t, 1H), 3.39 (s, 6H), 2.75 (d, 2H), 2.69 (t, 2H), 2.57-2.48 (m, 6H), 1.01 (ts, 6H).

### b) N-[2-(Diethylamino)ethyl]-N-(2,2-dimethoxyethyl)-3-[2-(1-naphthyl)ethoxy]propanamide

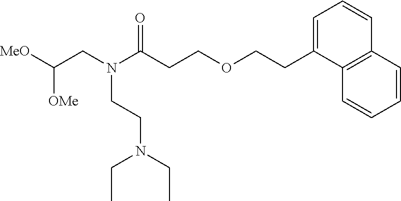

Oxalyl chloride (151 mL) was added dropwise over 45 minutes to a solution of 3-[2-(1-naphthyl)ethoxy]propanoic acid (389 g) (Example 7 step b)) in dichloromethane (2.1 L) and DMF (0.5 mL). The reaction mixture was stirred for a further 16 hours. The mixture was subsequently concentrated, redissolved in DCM (1.7 L) and added dropwise over 1.75 hours at 0° C. to a solution of N-(2,2-dimethoxyethyl)-N,N-diethylethane-1,2-diamine (325 g) and isopropyldiethylamine (551 mL) in DCM (1.7 L). The resulting mixture was stirred at room temperature for 3 hours, washed with aqueous saturated sodium bicarbonate solution (5×1 L), water (1.5 L) and dried over sodium sulphate and concentrated to give 650 g of the sub-titled compound.

m/e 431 (M+H⁺, 100%).

### c) N-[2-(Diethylamino)ethyl]-3-[2-(1-naphthyl)ethoxy]-N-(2-oxoethyl)propanamide

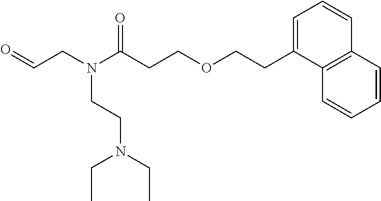

A solution of N-[2-(diethylamino)ethyl]-N-(2,2-dimethoxyethyl)-3-[2-(1-naphthyl)ethoxy]propanamide (93 g) in DCM (270 mL) was treated dropwise at 0° C. with trifluoroacetic acid (270 mL) over 1.5 hours. After the addition the reaction mixture was allowed to warm to room temperature and stirred for a further 1 hour. The reaction mixture was concentrated and the residue poured into aqueous saturated sodium bicarbonate solution (1800 mL, caution). The aqueous mixture was extracted with DCM (4×400 mL) and the combined extracts were dried over magnesium sulphate and concentrated. The residue was used directly in the following reaction.

### d) N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide dihydrobromide

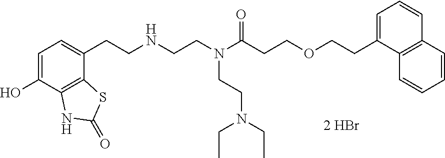

A suspension of 7-(2-amino-ethyl)-4-hydroxy-3H-benzothiazol-2-one hydrochloride (53 g) in dry NMP (216 mL) was heated to 60° C. and treated in one portion with a solution of NaOH (8.2 g) in methanol (102 mL). The bright orange suspension was cooled to room temperature and treated dropwise with a solution of N-[2-(diethylamino)ethyl]-3-[2-(1-naphthyl)ethoxy]-N-(2-oxoethyl)propanamide in dichloromethane (475 mL) over 20 minutes. The reaction was left to stir for 25 minutes. Sodium triacetoxyborohydride (91.5 g) was then added in portions over 20 minutes and the mixture stirred for a further 50 minutes. The reaction mixture was poured into water (1.8 L) and the acidic solution (pH5) was washed with tert. butyl methyl ether (TBME) (3×500 mL). The aqueous phase was basified to pH8 by the addition of solid potassium carbonate and extracted with dichloromethane (3×750 mL); the combined organic extracts were dried over magnesium sulphate and concentrated to give a dark oil. This was dissolved in ethanol (200 mL) and 48% aqueous hydrobromic acid (73 mL) was added. The solution was aged for 30 minutes then evaporated to dryness. The residue was triturated with ethanol (560 mL); the resultant solid was collected by filtration and dried in vacuo at 50° C. The sticky solid was suspended in boiling ethanol (100 mL) and filtered while hot. The collected solid was dried in vacuo at 50° C. This material was recrystallised from ethanol/water (3:1, 500 mL). After standing overnight the resultant solid was collected by filtration and washed with ice-cold ethanol (75 mL). Drying in vacuo at 50° C. for 24 hr afforded 57 g of the title compound.

### β₂-Adrenoceptor Agonist 2

### (BA2)

### N-[2-(Diethylamino)ethyl]-N-(2-{[2-(4-hydroxy-2-oxo-2,3-dihydro-1,3-benzothiazol-7-yl)ethyl]amino}ethyl)-3-[2-(3-chlorophenyl)ethoxy]propanamide dihydrobromide

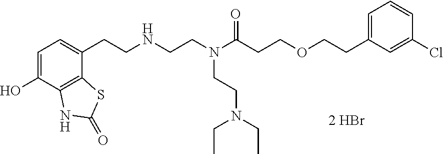

### a) tert-Butyl 3-[2-(3-chlorophenyl)ethoxy]propanoate

2-(3-chlorophenyl)ethanol (20 g) was treated with benzyltrimethylammonium hydroxide (Triton B®) (2.67 mL) and the resultant mixture was stirred in vacuo for 30 minutes. The mixture was then cooled to 0° C. and treated with t-butyl acrylate (17.40 g). The reaction was warmed to room temperature and stirred for 16 hours. The mixture was filtered through aluminium oxide (15 g) eluting with ether (75 mL). The collected filtrate was concentrated to give the sub-titled compound (34.40 g) as an oil.

¹H NMR (CDCl₃) δ 7.26-7.07 (m, 4H), 3.69-3.59 (m, 4H), 2.86-2.81 (t, 2H), 2.50-2.45 (t, 2H), 1.43 (s, 9H).

### b) 3-[2-(3-chlorophenyl)ethoxy]propanoic acid

tert-Butyl 3-[2-(3-chlorophenyl)ethoxy]propanoate (example 1a), 34.40 g) was dissolved in dichloromethane (150 mL) and treated with trifluoroacetic acid (50 mL). The mixture was stirred at room temperature for 3 hours, then concentrated in vacuo and azeotroped with dichloromethane (2×10 mL). The residue was taken up in dichlormethane (300 mL) and extracted with saturated sodium hydrogen carbonate (200 mL). The basic layer was washed with dichloromethane (20 mL) then acidified with 2M hydrochloric acid. The acidic layer was extracted with dichloromethane (2×200 mL). The organic layers were combined, washed with brine, dried over anhydrous magnesium sulphate, filtered and concentrated to yield the sub-titled compound (24.50 g) as an oil.

m/e 227 [M−H].

### c) N-[2-(Diethylamino)ethyl]-N-(2,2-dimethoxyethyl)-3-[2-(3-chlorophenyl)ethoxy]propanamide

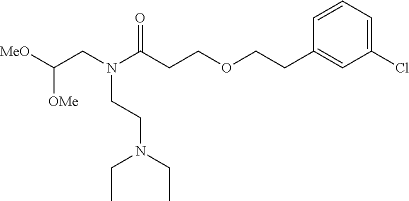

Oxalyl chloride (9.50 mL) was added dropwise over 45 minutes to a solution of 3-[2-(3-chlrophenyl)ethoxy]propanoic acid (22.50 g) (example 1b) in dichloromethane (120 ml) and DMF (0.5 mL). The reaction mixture was stirred for a further 16 hours. The mixture was subsequently concentrated, redissolved in DCM (1.7 L) and added dropwise over 1.75 hours at 0° C. to a solution of N-(2,2-dimethoxyethyl)-N,N-diethylethane-1,2-diamine (20.20 g) (example 16a) and isopropyldiethylamine (34.43 mL) in DCM (200 mL). The resulting mixture was stirred at room temperature for 16 hours, washed with aqueous saturated sodium bicarbonate solution (3×1 L), water (1.5 L) and dried over sodium sulphate and concentrated to give 39.50 g of the sub-titled compound.

m/e 415 (M+H⁺, 83%).

### d) N-[2-(Diethylamino)ethyl]-3-[2-(3-chlorophenyl)ethoxy]-N-(2-oxoethyl)propanamide

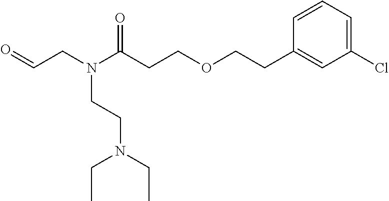

A solution of N-[2-(Diethylamino)ethyl]-N-(2,2-dimethoxyethyl)-3-[2-(3-chlorophenyl)ethoxy]propanamide (example 1c) (20 g) in DCM (500 mL) was treated dropwise at 0° C. with trifluoroacetic acid (50 mL) over 30 minutes. After the addition the reaction mixture was allowed to warm to room temperature and stirred for a further 1 hour. The reaction mixture was concentrated and the residue poured into aqueous saturated sodium bicarbonate solution (1800 mL, caution). The aqueous mixture was extracted with DCM (3×400 mL) and the combined extracts were dried over magnesium sulphate and concentrated. The residue was used directly in the following reaction.

### e) N-[2-(Diethylamino)ethyl]-N-(2-{[2-(4-hydroxy-2-oxo-2,3-dihydro-1,3-benzothiazol-7-yl)ethyl]amino}ethyl)-3-[2-(3-chlorophenyl)ethoxy]propanamide dihydrobromide

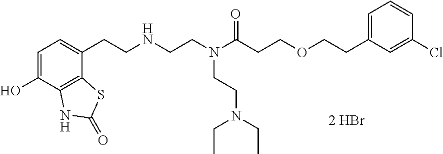

A suspension of 7-(2-amino-ethyl)-4-hydroxy-3H-benzothiazol-2-one hydrochloride (11.77 g) in dry NMP (50 mL) was heated to 65° C. and treated in one portion with a solution of NaOH (1.83 g) in methanol (23 mL). The bright orange suspension was cooled to room temperature and treated dropwise with a solution of N-[2-(diethylamino)ethyl]-3-[2-(3-chlorophenyl)ethoxy]-N-(2-oxoethyl)propanamide (example 1d) in dichloromethane (50 mL) over 30 minutes. The reaction was left to stir for 30 minutes. Sodium triacetoxyborohydride (20.33 g) was then added in portions over 20 minutes and the mixture stirred for a further 16 hours. The reaction mixture was poured into water (1.8 L), basified to pH8 by the addition of solid potassium carbonate and extracted with dichloromethane (2×500 mL); the combined organic extracts were dried over magnesium sulphate and concentrated to give a dark oil. The residue was purified by chromatography on silica with 10% (0.1% aqNH₃/MeOH)/DCM as eluent to give the sub-title compound as a brown oil. Yield (6.58 g). This was dissolved in ethanol (150 mL) and 48% aqueous hydrobromic acid (10 mL) was added. The solution was aged for 30 minutes then evaporated to dryness. The residue was triturated with ethanol (100 mL); the resultant solid was collected by filtration and dried in vacuo at 50. This material was recrystallised from ethanol/water (6:1, 500 mL); after standing overnight the resultant solid was collected by filtration and washed with ice-cold ethanol (75 mL). Drying in vacuo at 50° C. for 24 hr afforded 4.96 g of the title compound.

MS: APCI (+ve): 563 (M+1) 99.3% purity (T9505M).

1H NMR (DMSO, 90° C.), δ 11.75-11.73 (m, 1H), 10.08-10.06 (d, 1H), 8.65 (bs, 1H), 7.33-7.19 (m, 4H), 6.89-6.84 (t, 1H), 6.77-6.74 (m, 1H), 3.68-3.58 (m, 8H), 3.17-3.16 (m, 10H), 2.86-2.80 (m, 4H), 2.67-2.62 (m, 2H), 1.23-1.19 (t, 6H).

### Elemental Analysis

CHNS C, 46.54% (46.39); H, 5.75% (5.70); N, 7.94% (7.73); S, 4.46% (4.42).

### β₂-Adrenoceptor Agonist 3

### (BA3)

### 7-[(1R)-2-({2-[(3-{[2-(2-Chlorophenyl)ethyl]amino}propyl)thio]ethyl}amino)-1-hydroxyethyl]-4-hydroxy-1,3-benzothiazol-2(3H)-one dihydrobromide

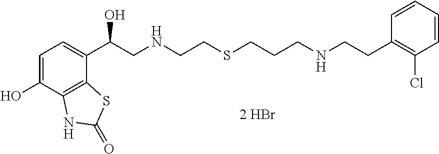

### a) 1-Chloro-2-[(E)-2-nitrovinyl]benzene

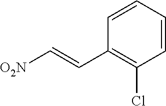

2-Chlorobenzaldehyde (ex Aldrich) (10.0 g) was mixed with nitromethane (26.05 g) and ammonium acetate (21.92 g) in acetic acid (200 mL), and the mixture was heated at reflux for 40 minutes. The mixture was allowed to cool to room temperature, and the majority of the acetic acid was removed in vacuo. The residue was dissolved in dichloromethane and washed with water, then potassium carbonate solution (×2), then water again. The organics were dried over anhydrous magnesium sulfate, filtered and evaporated to give the desired material, as an orange oil (12.83 g).

¹H NMR δ (CDCl₃) 8.41 (d, 1H), 7.62-7.57 (m, 2H), 7.52-7.48 (m, 1H), 7.43 (dt, 1H), 7.34 (ddd, 1H).

### b) 2-(2-Chlorophenyl)ethanamine

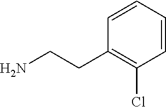

Aluminium hydride was prepared by the drop-wise addition of a solution of sulphuric acid (8.40 mL) in dry THF (60 mL) to a stirred solution of 1.0M lithium aluminium hydride in THF (314 mL), at 0-10° C., under a nitrogen atmosphere. After stirring at 5° C. for 30 minutes, a solution of 1-chloro-2-[(E)-2-nitrovinyl]benzene (12.83 g) in dry THF (160 mL) was added dropwise maintaining the internal temperature between 0° C. and 10° C. When the addition was complete the reaction was heated at reflux for 5 minutes. The mixture was allowed to cool to room temperature, then cooled to 0° C. and isopropanol (22 mL) carefully added dropwise maintaining the temperature below 20° C. 2M Sodium hydroxide (35 mL) was carefully added dropwise maintaining the temperature below 20° C. The mixture was stirred at room temperature for 30 minutes, then filtered through a layer of celite, which was then washed with THF (×3). The filtrate was evaporated to dryness. The residue was purified using silica column chromatography, using ethyl acetate to load the material, then 10% triethylamine in ethyl acetate, followed by 10% triethylamine in 45% ethanol:45% ethyl acetate as the eluents, to give the desired material (4.66 g). ¹H NMR δ(CDCl₃) 7.36 (dd, 1H), 7.25-7.13 (m, 3H), 2.98 (dt, 2H), 2.91-2.87 (m, 2H).

### c) tert-Butyl[2-(2-chlorophenyl)ethyl]carbamate

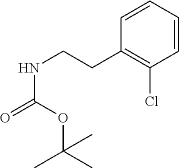

To a stirred solution of 2-(2-chlorophenyl)ethanamine (25.57 g) and triethylamine (22.87 mL) in dry THF (300 mL) was added a solution of di-tert-butyl dicarbonate (35.85 g) in dry THF (50 mL) over 10 minutes, at ambient temperature, under a nitrogen atmosphere. The reaction mixture was stirred at room temperature for 3 hours. The solvents were removed in vacuo to give the desired material, as a yellow oil (42.0 g).

¹H NMR δ(CDCL3) 7.35 (d, 1H), 7.25-7.14 (m, 3H), 4.57 (s, 1H), 3.43-3.35 (m, 2H), 2.95 (t, 2H), 1.43 (d, 9H).

### d) tert-Butyl allyl[2-(2-chlorophenyl)ethyl]carbamate

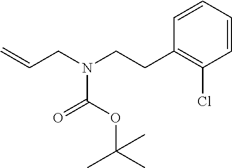

To a suspension of sodium hydride (60% in mineral oil) (7.23 g), which had been washed with ether (×3), in dry DMF (200 mL) was added a solution of tert-butyl[2-(2-chlorophenyl)ethyl]carbamate (42.0 g) in dry DMF (50 mL), over a 15 minute period, at 35° C., under a nitrogen atmosphere. When the addition was complete, the mixture was stirred at 50° C. for 90 minutes. The mixture was allowed to cool to room temperature, then allyl bromide (15.63 mL) was added slowly, keeping the temperature at 25° C., using external cooling. The mixture was stirred at room temperature for 2 hours, then diluted with water and extracted with ethyl acetate (×3). The organics were combined, washed with water, dried over anhydrous magnesium sulfate, filtered and evaporated. The residue was purified using silica column chromatography, loading with 1% ethyl acetate in isohexane, then using isohexane with ethyl acetate (0%, 1%, 2%, %5) as the eluents to give the desired material (27.0 g). There were several mixed fractions, so these were combined, and re-purified using silica column chromatography, as above, to give a further 4 g of desired material. Both crops of product were combined to give 31.0 g in total. ¹H NMR δ (CDCl₃) 7.36-7.31 (m, 1H), 7.21-7.12 (m, 3H), 5.83-5.68 (m, 1H), 5.17-5.05 (m, 2H), 3.86-3.66 (m, 2H), 3.41 (t, 2H), 3.03-2.90 (m, 2H), 1.43 (s, 9H). HPLC: 95.90% @ 220 nm [M+H-Boc]+=196.1 (Calc=295.1339) (multimode+).

### e) tert-Butyl[2-(2-chlorophenyl)ethyl]{3-[(2-hydroxyethyl)thio]propyl}carbamate

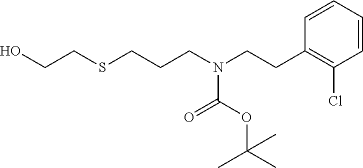

tert-Butyl allyl[2-(2-chlorophenyl)ethyl]carbamate (31.0 g) was mixed with 2-mercaptoethanol (7.37 mL), and AlBN (1.15 g), and stirred at 65° C. for 45 minutes. The mixture was cooled and more mercaptoethanol (1 mL) and AlBN (200 mg) added. The mixture was then heated at 65° C. for a further 30 minutes. The material was purified by silica column chromatography, loading the material in 20% ethyl acetate in isohexane, then eluting with 20% ethyl acetate in isohexane, changing to 50%, to give the desired material (31.94 g).

¹H NMR δ(CDCl₃) 7.38-7.32 (m, 1H), 7.22-7.13 (m, 3H), 3.75-3.68 (m, 2H), 3.41 (t, 2H), 3.32-3.14 (m, 2H), 3.03-2.91 (m, 2H), 2.72 (t, 2H), 2.54-2.36 (m, 2H), 1.85-1.71 (m, 2H), 1.42 (s, 9H).

HPLC: 92.31% @ 220 nm [M+H-Boc]+=274.1 (Calc=373.1478) (multimode+).

### f) tert-Butyl[2-(2-chlorophenyl)ethyl]{3-[(2-oxoethyl)thio]propyl}carbamate

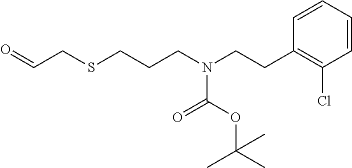

Sulfur trioxide:pyridine complex (30.52 g) was dissolved in DMSO (200 mL) and stirred at room temperature, under a nitrogen atmosphere, for 15 minutes. DCM (100 mL) was added, followed by a solution of tert-butyl[2-(2-chlorophenyl)ethyl]{3-[(2-hydroxyethyl)thio]propyl}carbamate (23.9 g) and Hunigs base (63.5 mL) in DCM (160 mL), which was added in one portion (exotherm). The resulting mixture was stirred at ambient temperature for 15 minutes. The reaction mixture was diluted with ethyl acetate, washed with water, then 1N HCl, then saturated sodium bicarbonate solution, dried over anhydrous magnesium sulfate, filtered and the solvents removed in vacuo. The material was purified by silica column chromatography eluting with 20% ethyl acetate in isohexane to give the desired material (12.43 g).

¹H NMR δ(CDCl₃) 9.46 (t, 1H), 7.36-7.32 (m, 1H), 7.21-7.13 (m, 3H), 3.40 (t, 2H), 3.29-3.13 (m, 4H), 3.02-2.90 (m, 2H), 2.45-2.34 (m, 2H), 1.82-1.69 (m, 2H), 1.49-1.36 (m, 9H).

### g) tert-Butyl[2-(2-chlorophenyl)ethyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate

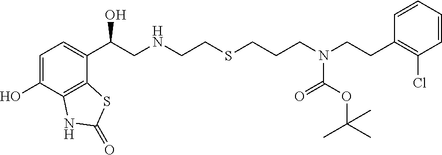

The tert-butyl[2-(2-chlorophenyl)ethyl]{3-[(2-oxoethyl)thio]propyl}carbamate (11.32 g) was dissolved in a mixture of methanol (200 mL) and acetic acid (1.74 ml). 7-[(1R)-2-amino-1-hydroxyethyl]-4-hydroxy-1,3-benzothiazol-2(3H)-one hydrochloride (8.0 g) was added to the solution, and the mixture stirred at room temperature, under a nitrogen atmosphere, for 1 hour. Sodium cyanoborohydride (1.92 g) was added and the mixture stirred for a further 2 hours. The solvents were removed in vacuo, and the residue diluted with water, basified with 0.880 aqueous ammonia, and extracted with ethyl acetate (×3) (filtered through celite during extraction). The organics were combined, washed with brine, dried over anhydrous sodium sulfate, filtered and evaporated to give a brown residue (15.5 g). The material was purified using silica column chromatography, using DCM with MeOH (2%, 5%, 10%, 20% and 30%, all with 1% 0.880 aq NH₃) as the eluent, to give the desired material (6.67 g) (38% yield)

¹H NMR δ(DMSO) 7.43-7.38 (m, 1H), 7.30-7.21 (m, 3H), 6.86 (d, 1H), 6.69 (d, 1H), 4.56 (dd, 1H), 3.23-3.10 (m, 2H), 2.88 (t, 2H), 2.71-2.48 (m, 8H), 2.46-2.39 (m, 2H), 1.72-1.62 (m, 2H), 1.40-1.22 (m, 9H).

HPLC: 97.46% @ 220 nm [M+H]+=582.1 (Calc=582.1863) (multimode+).

### h) 7-[(1R)-2-({2-[(3-{[2-(2-Chlorophenyl)ethyl]amino}propyl)thio]ethyl}amino)-1-hydroxyethyl]-4-hydroxy-1,3-benzothiazol-2(3H)-one dihydrobromide

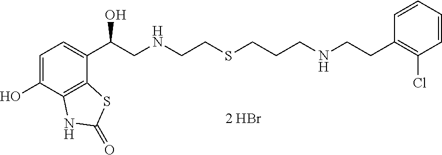

To a stirred suspension of the Boc compound from part g) (5.93 g) in DCM (20 mL) was added trifluoroacetic acid (20 mL) at 0° C., and the resulting mixture was stirred under nitrogen for 30 minutes. The mixture was diluted with toluene, and solvents removed, then azeotroped with toluene (×2). The residue was dissolved in acetonitrile, acidified with 48% aq HBr and concentrated in vacuo (not to dryness). The mixture was further diluted with acetonitrile and the precipitated solid collected by filtration, washed with acetonitrile and dried under vacuum to give 6.35 g. A 3.8% impurity was present (isomer from part e)), so the material was redissolved in a 1:1 mixture of acetonitrile:water and purified using prep HPLC (Sunfire 30×80 mm C8 column; NH₄OAc buffer; acetonitrile 5-50% over 10 minutes). The resultant material was dried overnight in a dessicator at 10 mbar over KOH and H₂SO₄. The resulting di-acetate salt was dissolved in water and basified with 0.880 aq ammonia. A white gum formed, so the aqueous was decanted off, and the gum dried in vacuo to give the free base (4.11 g). This was dissolved in hot ethanol, and the solution was filtered, then allowed to cool to room temperature. The solution was acidified with 48% aq. HBr and left to crystallize. The white solid was collected by filtration, washed with ethanol and dried in vacuo to give 3.81 g Crop 1.

¹H NMR δ(DMSO) 11.67 (s, 1H), 10.15 (s, 1H), 8.70 (s, 4H), 7.50-7.30 (m, 4H), 6.94 (d, 1H), 6.78 (d, 1H), 6.45 (s, 1H), 4.96-4.90 (m, 1H), 3.22-3.02 (m, 10H), 2.86-2.76 (m, 2H), 2.66 (t, 2H), 1.91 (quintet, 2H).

HPLC: 99.63% @ 220 nm [M+H]+=482 (calc=482.1339) (MultiMode+).

### Elemental Analysis:

| | | C | H | N | S |
|---|---|---|---|---|---|
| Calculated: | | 41.04 | 4.70 | 6.53 | 9.96 |
| Found: | 1: | 41.07 | 4.69 | 6.67 | 9.72 |
| | 2: | 41.08 | 4.68 | 6.74 | 9.67 |
| | 3: | 40.96 | 4.68 | 6.75 | 9.67 |

The mother liquors were evaporated to dryness then triturated with acetonitrile. The solid was collected by filtration to give 719 mg Crop 2 (4.53 g total).

¹H NMR δ(DMSO) 11.67 (s, 1H), 10.15 (s, 1H), 8.80-8.60 (m, 4H), 7.50-7.29 (m, 4H), 6.94 (d, 1H), 6.78 (d, 1H), 6.45 (s, 1H), 4.96-4.89 (m, 1H), 3.22-3.00 (m, 10H), 2.85-2.76 (m, 2H), 2.66 (t, 2H), 1.90 (quintet, 2H).

HPLC: 99.20% @ 220 nm [M+H]+=482 (calc=482.1339) (MultiMode+).

### Elemental Analysis:

| | | C | H | N | S |
|---|---|---|---|---|---|
| Calculated: | | 41.04 | 4.70 | 6.53 | 9.96 |
| Found: | 1: | 40.90 | 4.69 | 6.78 | 9.60 |
| | 2: | 41.01 | 4.70 | 6.83 | 9.60 |
| | 3: | 40.97 | 4.69 | 6.76 | 9.63 |

### Biological Activity of β₂-Adrenoceptor Agonists

Adrenergic β2 Mediated cAMP Production

### Cell Preparation

H292 cells were grown in 225 cm2 flasks incubator at 37° C., 5% CO₂ in RPMI medium containing, 10% (v/v) FBS (foetal bovine serum) and 2 mM L-glutamine.

### Experimental Method

Adherent H292 cells were removed from tissue culture flasks by treatment with Accutase™ cell detachment solution for 15 minutes. Flasks were incubated for 15 minutes in a humidified incubator at 37° C., 5% CO₂. Detached cells were re-suspended in RPMI media (containing 10% (v/v) FBS and 2 mM L-glutamine) at 0.05×10⁶ cells per mL. 5000 cells in 100 μL were added to each well of a tissue-culture-treated 96-well plate and the cells incubated overnight in a humidified incubator at 37° C., 5% CO₂. The culture media was removed and cells were washed twice with 100 μL assay buffer and replaced with 50 μL assay buffer (HBSS solution containing 10 mM HEPES pH7.4 and 5 mM glucose). Cells were rested at room temperature for 20 minutes after which time 25 μL of rolipram (1.2 mM made up in assay buffer containing 2.4% (v/v) dimethylsulphoxide) was added. Cells were incubated with rolipram for 10 minutes after which time Compound A was added and the cells were incubated for 60 minutes at room temperature. The final rolipram concentration in the assay was 300 μM and final vehicle concentration was 1.6% (v/v) dimethylsulphoxide. The reaction was stopped by removing supernatants, washing once with 100 μL assay buffer and replacing with 50 μL lysis buffer. The cell monolayer was frozen at −80° C. for 30 minutes (or overnight).

AlphaScreen™ cAMP Detection

The concentration of cAMP (cyclic adenosine monophosphate) in the cell lysate was determined using AlphaScreen™ methodology. The frozen cell plate was thawed for 20 minutes on a plate shaker then 10 μL of the cell lysate was transferred to a 96-well white plate. 40 μL of mixed AlphaScreen™ detection beads pre-incubated with biotinylated cAMP, was added to each well and the plate incubated at room temperature for 10 hours in the dark. The AlphaScreen™ signal was measured using an EnVision spectrophotometer (Perkin-Elmer Inc.) with the recommended manufacturer's settings. cAMP concentrations were determined by reference to a calibration curve determined in the same experiment using standard cAMP concentrations. A concentration response curve for Compound A was constructed and data was fitted to a four parameter logistic equation to determine both the pEC₅₀ and Intrinsic Activity. Intrinsic Activity was expressed as a fraction relative to the maximum activity determined for formoterol in each experiment. Result are in Table 1.

### Selectivity Assays

### Adrenergic α1D

### Membrane Preparation

Membranes were prepared from human embryonic kidney 293 (HEK293) cells expressing recombinant human α1_{D} receptor. These were diluted in Assay Buffer (50 mM HEPES, 1 mM EDTA, 0.1% gelatin, pH 7.4) to provide a final concentration of membranes that gave a clear window between maximum and minimum specific binding.

### Experimental Method

Assays were performed in U-bottomed 96-well polypropylene plates. 10 μL [³H]-prazosin (0.3 nM final concentration) and 10 μL of Compound A (10× final concentration) were added to each test well. For each assay plate 8 replicates were obtained for [³H]-prazosin binding in the presence of 10 μL vehicle (10% (v/v) DMSO in Assay Buffer; defining maximum binding) or 10 μL BMY7378 (10 μM final concentration; defining non-specific binding (NSB)). Membranes were then added to achieve a final volume of 100 μL. The plates were incubated for 2 hours at room temperature and then filtered onto PEI coated GF/B filter plates, pre-soaked for 1 hour in Assay Buffer, using a 96-well plate Tomtec cell harvester. Five washes with 250 μL wash buffer (50 mM HEPES, 1 mM EDTA, pH 7.4) were performed at 4° C. to remove unbound radioactivity. The plates were dried then sealed from underneath using Packard plate sealers and MicroScint-O (50 μL) was added to each well. The plates were sealed (TopSeal A) and filter-bound radioactivity was measured with a scintillation counter (TopCount, Packard BioScience) using a 3-minute counting protocol.

Total specific binding (B₀) was determined by subtracting the mean NSB from the mean maximum binding. NSB values were also subtracted from values from all other wells. These data were expressed as percent of B₀. Compound concentration-effect curves (inhibition of [³H]-prazosin binding) were determined using serial dilutions typically in the range 0.1 nM to 10 μM. Data was fitted to a four parameter logistic equation to determine the compound potency, which was expressed as pIC50 (negative log molar concentration inducing 50% inhibition of [³H]-prazosin binding). Results are shown in Table 1 below.

### Adrenergic β1

### Membrane Preparation

Membranes containing recombinant human adrenergic beta 1 receptors were obtained from Euroscreen. These were diluted in Assay Buffer (50 mM HEPES, 1 mM EDTA, 120 mM NaCl, 0.1% gelatin, pH 7.4) to provide a final concentration of membranes that gave a clear window between maximum and minimum specific binding.

### Experimental Method

Assays were performed in U-bottomed 96-well polypropylene plates. 10 μL [¹²⁵I]-Iodocyanopindolol (0.036 nM final concentration) and 10 μL of Compound A (10× final concentration) were added to each test well. For each assay plate 8 replicates were obtained for [¹²⁵I]-Iodocyanopindolol binding in the presence of 10 μL vehicle (10% (v/v) DMSO in Assay Buffer; defining maximum binding) or 10 μL Propranolol (10 μM final concentration; defining non-specific binding (NSB)). Membranes were then added to achieve a final volume of 100 μL. The plates were incubated for 2 hours at room temperature and then filtered onto PEI coated GF/B filter plates, pre-soaked for 1 hour in Assay Buffer, using a 96-well plate Tomtec cell harvester. Five washes with 250 μL wash buffer (50 mM HEPES, 1 mM EDTA, 120 mM NaCl, pH 7.4) were performed at 4° C. to remove unbound radioactivity. The plates were dried then sealed from underneath using Packard plate sealers and MicroScint-O (50 μL) was added to each well. The plates were sealed (TopSeal A) and filter-bound radioactivity was measured with a scintillation counter (TopCount, Packard BioScience) using a 3-minute counting protocol.

Total specific binding (B₀) was determined by subtracting the mean NSB from the mean maximum binding. NSB values were also subtracted from values from all other wells. These data were expressed as percent of B₀. Compound concentration-effect curves (inhibition of [¹²⁵I]-Iodocyanopindolol binding) were determined using serial dilutions typically in the range 0.1 nM to 10 μM. Data was fitted to a four parameter logistic equation to determine the compound potency, which was expressed as pIC₅₀ (negative log molar concentration inducing 50% inhibition of [¹²⁵I]-Iodocyanopindolol binding). Results are shown in Table 1 below.

### Dopamine D2

### Membrane Preparation

Membranes containing recombinant human Dopamine Subtype D2s receptors were obtained from Perkin Elmer. These were diluted in Assay Buffer (50 mM HEPES, 1 mM EDTA, 120 mM NaCl, 0.1% gelatin, pH 7.4) to provide a final concentration of membranes that gave a clear window between maximum and minimum specific binding.

### Experimental Method

Assays were performed in U-bottomed 96-well polypropylene plates. 30 μL [³H]-spiperone (0.16 nM final concentration) and 30 μL of Compound A (10× final concentration) were added to each test well. For each assay plate 8 replicates were obtained for [³H]-spiperone binding in the presence of 30 μL vehicle (10% (v/v) DMSO in Assay Buffer; defining maximum binding) or 30 μL Haloperidol (10 μM final concentration; defining non-specific binding (NSB)). Membranes were then added to achieve a final volume of 300 μL. The plates were incubated for 2 hours at room temperature and then filtered onto PEI coated GF/B filter plates, pre-soaked for 1 hour in Assay Buffer, using a 96-well plate Tomtec cell harvester. Five washes with 250 μL wash buffer (50 mM HEPES, 1 mM EDTA, 120 mM NaCl, pH 7.4) were performed at 4° C. to remove unbound radioactivity. The plates were dried then sealed from underneath using Packard plate sealers and MicroScint-O (50 μL) was added to each well. The plates were sealed (TopSeal A) and filter-bound radioactivity was measured with a scintillation counter (TopCount, Packard BioScience) using a 3-minute counting protocol.

Total specific binding (B₀) was determined by subtracting the mean NSB from the mean maximum binding. NSB values were also subtracted from values from all other wells. These data were expressed as percent of B₀. Compound concentration-effect curves (inhibition of [³H]-spiperone binding) were determined using serial dilutions typically in the range 0.1 nM to 10 μM. Data was fitted to a four parameter logistic equation to determine the compound potency, which was expressed as pIC₅₀ (negative log molar concentration inducing 50% inhibition of [³H]-spiperone binding). Results are shown in Table 3.

### Table 3

TABLE 3

| Com- | β2 | β2 | α1 | β1 | D2 |
|---|---|---|---|---|---|
| pound | pEC50 | Int Act | bind pIC50 | bind p IC50 | bind pIC50 |
| BA1 | 8.2 | 0.8 | 6.6 | <5 | 6.1 |
| BA2 | 8.3 | 0.7 | <6.1 | <5 | 5.6 |
| BA3 | 9.2 | 0.8 | 7.6 | 6.9 | 5.8 |

### In Vitro Combination Model

Evaluation of Compound Activity on Isolated Tracheal Rinds from Guinea-Pig Preconstricted with Methacholine.

The following protocol may be used to evaluate the effects of a muscarinic M3 receptor antagonist according to the present invention in combination with a β2-agonist.

Addition of β2-adrenoceptor agonists and/or muscarinic M3 receptor antagonists causes relaxation of isolated guinea-pig tracheal rings precontracted with the muscarinic agonist, methacholine. Male albino Dunkin Hartley guinea-pigs (300-350 g) are killed by cervical dislocation and the trachea excised. Adherent connective tissue is removed and the trachea cut into ring segments (2-3 mm wide). These are suspended in 10 mL organ baths containing a modified Krebs solution composition (mM): NaCl 117.56, KCl 5.36, NaH₂P0₄ 1.15, MgS0₄ 1.18, glucose 11.10, NaHCO₃ 25.00 and CaCl₂ 2.55. This is maintained at 37° C. and continually gassed with 5% CO₂ in O₂, Indomethacin (2.8 μM), corticosterone (10 μM), ascorbate (1 mM), CGP20712A (1 μM) and phentolamine (3 μM) are added to the Krebs solution: indomethacin to prevent development of smooth muscle tone due to the synthesis of cyclooxygenase products, corticosterone to inhibit the uptake 2 process, ascorbate to prevent catecholamine oxidation and CGP20712A and phentolamine to avoid any complicating effects of β1- and α-adrenoceptor activation respectively. The tracheal rings are suspended between two stainless steel hooks, one attached to an isometric force transducer and the other to a stationary support in the organ bath. Changes in isometric force are recorded. Acetyl-β-methylcholine chloride (Methacholine), Indomethacin, Corticosterone-21-acetate, Phentolamine hydrochloride, Ascorbic acid, CGP20712A methanesulphate are obtained from the Sigma Chemical Company. Indomethacin is dissolved in 10% w/v Na₂CO₃, corticosterone 21-acetate in ethanol and other compounds in DMSO. The muscarinic antagonist and formoterol are diluted in Krebs prior to adding to tissues and the level of DMSO in the bath was <0.1%.

At the beginning of each experiment a force of 1.0 g·wt. is applied to the tissues and this is reinstated over a 30 min equilibration period until it remained steady. Tissues are then exposed to 1 μM of the muscarinic agonist, methacholine, to assess tissue viability. Tissues are washed by exchanging the bathing Krebs solution three times. After 30 minutes the tissues are again precontracted with 1 μM methacholine. When the contraction has reached a plateau, 1 nM Formoterol, 10 nM of the muscarinic antagonist or a combination of both is added to the bathing media and left for 60 minutes.

Data are collected using the ADInstruments Chart5 for windows software, the tension generated is measured before addition of methacholine and after its response has reached plateau. The response to the mucorinic antagonist and/or formoterol is measured at 10 minute intervals following their addition. All responses are expressed as percentage inhibition of the methacholine-induced contraction.

### In Vivo Combination Model

### Evaluation of Lung Function in Anaesthetised Guinea Pigs.

The following protocol may be used to evaluate the effects of a muscarinic M3 receptor antagonist according to the present invention in combination with a β2-agonist.

Male Dunkin-Hartley guinea pigs (300-600 g) are weighed and dosed with vehicle (0.05M phosphate, 0.1% Tween 80, 0.6% saline, pH 6) or compound via the intratracheal route under recoverable gaseous anaesthesia (5% halothane in oxygen). Animals are dosed with compound or vehicle two hours prior to the administration of methacholine. Guinea pigs are anaesthetised with pentobarbitone (1 mL/kg of 60 mg/mL solution i.p.) approximately 30 minutes prior to the first bronchoconstrictor administration. The trachea is cannulated and the animal ventilated using a constant volume respiratory pump (Harvard Rodent Ventilator model 683) at a rate of 60 breath/min and a tidal volume of 5 mL/kg. A jugular vein is cannulated for the administration of methacholine or maintenance anaesthetic (0.1 mL of pentobarbitone solution, 60 mg/mL, as required).

The animals are transferred to a Flexivent System (SCIREQ, Montreal, Canada) in order to measure airway resistance. The animals are ventilated (quasi-sinusoidal ventilation pattern) at 60 breaths/min at a tidal volume of 5 mL/kg. A positive end expiratory pressure of 2-3 cm H₂O was applied. Respiratory resistance is measured using the Flexivent “snapshot” facility (1 second duration, 1 Hz frequency). Once a stable baseline resistance value has been obtained the animals are given methacholine in ascending doses (0.5, 1, 2, 3 and 5 μg/kg, i.v) at approximately 4-minute intervals via the jugular catheter. After each administration of bronchoconstrictor the peak resistance value is recorded. Guinea pigs are euthanised with approximately 1.0 mL pentobarbitone sodium (Euthatal) intravenously after the completion of the lung function measurements.

Percentage bronchoprotection produced by the compound is calculated at each dose of brochoconstrictor as follows:

$$\% bronchoprotection = \frac{\% {changeR}_{veh} - \% {changeR}_{cmpd}}{\% {changeR}_{veh}}$$

Where % change Rᵥₑₕ is the mean of the maximum percentage change in airway resistance in the vehicle treated group.

1. A pharmaceutical product comprising, in combination, a first active ingredient which is a muscarinic antagonist selected from:
(R)-1-[5-((R)-Cyclohexyl-hydroxy-phenyl-methyl)-[1,3,4]oxadiazol-2-ylmethyl]-3-(4-fluoro-phenoxy)-1-azonia-bicyclo[2.2.2]octane X;
(R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane X;
(R)-3-(3-Fluoro-4-methyl-phenoxy)-1-[3-(hydroxy-diphenyl-methyl)-isoxazol-5-ylmethyl]-1-azonia-bicyclo[2.2.2]octane X;
(R)-3-(3-Fluoro-phenylsulfanyl)-1-[3-(hydroxy-diphenyl-methyl)-isoxazol-5-ylmethyl]-1-azonia-bicyclo[2.2.2]octane X;
(R)-1-[3-((R)-Cyclohexyl-hydroxy-phenyl-methyl)-isoxazol-5-ylmethyl]-3-(4-fluoro-phenoxy)-1-azonia-bicyclo[2.2.2]octane X;
wherein X represents a pharmaceutically acceptable anion of a mono or polyvalent acid, and a second active ingredient which is a β₂-adrenoceptor agonist.

2. A product according to claim 1 wherein the first active ingredient is a muscarinic antagonist, which is a bromide salt.

3. A product according to claim 1 or claim 2, wherein the β₂-adrenoceptor agonist is formoterol.

4. A product according to claim 1 or claim 2 wherein the β₂-adrenoceptor agonist is selected from:
N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide,
N-[2-(Diethylamino)ethyl]-N-(2-{[2-(4-hydroxy-2-oxo-2,3-dihydro-1,3-benzothiazol-7-yl)ethyl]amino}ethyl)-3-[2-(3-chlorophenyl)ethoxy]propanamide, and
7-[(1R)-2-({2-[(3-{[2-(2-Chlorophenyl)ethyl]amino}propyl)thio]ethyl}amino)-1-hydroxyethyl]-4-hydroxy-1,3-benzothiazol-2(3H)-one,
or a pharmaceutically acceptable salt thereof.

5. A product according to claim 1 or claim 2 wherein the β₂-adrenoceptor agonist is N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide or a pharmaceutically acceptable salt thereof.

6. A product according to claim 1 wherein the muscarinic receptor antagonist is (R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane X, wherein X represents a pharmaceutically acceptable anion of a mono or polyvalent acid, and the β₂-adrenoceptor agonist is N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide or a pharmaceutically acceptable salt thereof.

7. A product according to claim 1 wherein the muscarinic receptor antagonist is (R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane X, wherein X represents a pharmaceutically acceptable anion of a mono or polyvalent acid, and the β₂-adrenoceptor agonist is N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide or a pharmaceutically acceptable salt thereof.

8. Use of a product according to any one of claims 1 to 7 in the manufacture of a medicament for the treatment of a respiratory disease.

9. Use according to claim 7, wherein the respiratory disease is chronic obstructive pulmonary disease.

10. A method of treating a respiratory disease, which method comprises simultaneously, sequentially or separately administering:
(a) a (therapeutically effective) dose of a first active ingredient which is a muscarinic receptor antagonist as defined in claim 1 or claim 2; and
(b) a (therapeutically effective) dose of a second active ingredient which is a β₂-adrenoceptor agonist; to a patient in need thereof.

11. A kit comprising a preparation of a first active ingredient which is a muscarinic receptor antagonist as defined in claim 1 or claim 2, and a preparation of a second active ingredient which is a β₂-adrenoceptor agonist and optionally instructions for the simultaneous, sequential or separate administration of the preparations to a patient in need thereof.

12. A pharmaceutical composition comprising, in admixture, a first active ingredient which is a muscarinic receptor antagonist as defined in claim 1 or claim 2 and a second active ingredient which is a β₂-adrenoceptor agonist.
